(19) United States
(12) Patent Application Publication
(10) Pub. No.: US 2026/0357325 A1
(43) Pub. Date: Nov. 12, 2026

(54) DNA-Binding Compounds for Detecting Nucleic Acids

(71) Applicant: AAT Bioquest, Inc., Pleasanton, CA (US)

(72) Inventors: Zhenjun DIWU, Danville, CA (US); Jixiang LIU, Pleasanton, CA (US); Qinglin MENG, Foster City, CA (US); Deven PATEL, Santa Clara, CA (US); Jinfang LIAO, Danville, CA (US)

(21) Appl. No.: 19/178,790

(22) Filed: Apr. 14, 2025

(51) Int. Cl. C12Q 1/6806; C07C 233/36; C07D 207/34; C07D 235/20; C07D 417/06; C07F 15/00

(52) U.S. Cl. CPC C12Q 1/6806; C07C 233/36; C07D 207/34; C07D 235/20; C07D 417/06; C07F 15/0093; C07C 2603/24

(57) ABSTRACT

Nucleic acid detecting compounds having base-reactive groups (BRM) are used for detecting nucleic acids, particularly for viability PCR-based detection of DNA, including compounds having the following formula or its tautomeric formula or a salt thereof:

[BRM1-L1]ₘ-NBM-[L2-BRM2]ₙ

wherein BRM1 and BRM2 are independently a nucleic acid base-reactive moiety, NBM is a nucleic acid binding moiety, L1 and L2 are independently a linker, and m and n are independently an integer from 0 to 4, wherein the sum of m and n is from 1 to 4.

### FIELD

The present disclosure relates to nucleic acid-reactive compounds for detecting nucleic acids, including DNA. The compounds have applications in the fields of molecular biology, particularly with respect to viability PCR-based assays and tests. The compounds of present disclosure can be used with viability PCR for detecting bacteria, viruses and other biological samples. The compounds of present disclosure are extremely robust without the UV irradiation that is required for the commonly used PMA-based viability PCR tests.

### BACKGROUND

In many fields of life sciences research, including biological, biomedical, genetic, fermentation, aquaculture, agricultural, forensic and environmental research, there is a need to detect nucleic acids (in particular DNA). In general, the detection methodology should be fast, sensitive, and selective. Such applications require to detect minute amounts of nucleic acids in a variety of samples.

Polymerase chain reaction (PCR) is an important means for investigating microorganisms, such as bacteria and viruses. However, the regular PCR is limited by its inability to differentiate DNA originating from viable, metabolically active versus non-viable, inactive bacteria. This drawback has led to concerns that PCR-based diagnosis of bacteria and viruses are confounded by the presence of relic DNA from non-viable bacteria or viruses (A. Lee et al, Front Cell Infect Microbiol, 2022, 12).

Propidium monoazide (PMA)-based viability PCR (VPCR) has been widely used to overcome this selectivity drawback in detecting bacteria and viruses that are involved in biodefense, food safety, in vitro diagnostics (IVD), pathology, forensics and drug discovery. PMA only penetrates cells with compromised cell membranes (non-viable cells) and reacts with DNA, rendering it unable to be amplified by PCR. PMA-based PCR has been shown to differentiate between non-viable and viable bacteria in many settings (U.S. Pat. No. 10,570,463; A. Lee et al, Front Cell Infect Microbiol, 2022, 12; Z. Zhang et al., J. Dairy Sci. 2015, 98, 1625-1633).

Although PMA has been shown to differentiate between non-viable and viable bacteria in various settings, its effectiveness requires strong UV irradiation of samples. UV irradiation is well known to cause DNA damages and other detrimental damages to biological samples to be analyzed, thus resulting in inaccurate results (M. Kuick et al, Int J Mol Sci, 2020, 21(19), 7264; S. Kaur et al, Anal Chem, 2024, 2914-2921). In addition, UV irradiation requires operations carried out in the dark, which makes the sample analysis tedious and inconvenient with low throughput.

### SUMMARY

The present disclosure provides nucleic acid detecting compounds that include a nucleic acid binding moiety configured adjacent to one, two or more base-reactive moieties capable of forming covalent linkages with a target nucleic acid to which the compound is bound. The nucleic acid detecting compounds can be conveniently used with viability PCR assays for detecting bacteria, viruses and other biological samples. In some embodiments, the compounds of the present disclosure are extremely robust without the need for UV irradiation that is required for operation of the existing PMA-based viability PCR tests.

In some embodiments, the DNA-binding compounds are of Formula 1:

[BRM1-L1]ₘ-NBM-[L2-BRM2]ₙ

or a salt thereof, wherein:
BRM1 and BRM2 are independently a nucleic acid base-reactive moiety;
NBM is a nucleic acid binding moiety;
L1 and L2 are independently a linker; and
m and n are independently an integer from 0 to 4, wherein the sum of m and n is from 1 to 4.

In some embodiments, the DNA-binding compounds are of the following formula or its tautomeric formula:

{[BRM1-L1]ₘ-NBM-[L2-BRM2]ₙ}W

wherein BRM1 and BRM2 are independently a nucleic acid base-reactive moiety; NBM is a nucleic acid binding moiety; L1 and L2 are independently a linker; m and n are independently an integer from 0 to 4; and W is an optional physiological counter ion.

In some embodiments, the DNA-binding compounds are of Formula 2:

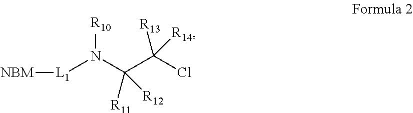

or a salt thereof, wherein:
NBM is selected from an intercalator and a minor groove binding moiety, and
R₁₀-R₁₄ are independently selected from H, an alkyl, an alkoxy, an aryloxy, an aryl, and a heteroaryl; and
L1 is a linker comprising 1-100 non-hydrogen atoms selected from C, N, O, P, and S, further comprising a plurality of single, double, triple or aromatic carbon-carbon bonds, carbon-nitrogen bonds, nitrogen-nitrogen bonds, carbon-oxygen bonds, carbon-sulfur bonds, phosphorus-oxygen bonds, phosphorus-nitrogen bonds, and combination thereof.

In some embodiments, the DNA-binding compounds are of Formula 3:

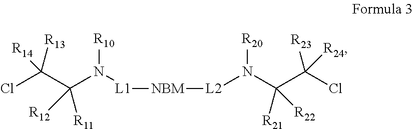

or a salt thereof, wherein:
NBM is selected from an intercalator and a minor groove binding moiety;
R₁₀-R₁₄ and R₂₀-R₂₄ are independently selected from H, an alkyl, an alkoxy, an aryloxy, an aryl, and a heteroaryl; and
L1 and L2 are each independently a covalent linkage having 1-100 non-hydrogen atoms selected from C, N, O, P, and S, further comprising a plurality of single, double, triple or aromatic carbon-carbon bonds, carbon-nitrogen bonds, nitrogen-nitrogen bonds, carbon-oxygen bonds, carbon-sulfur bonds, phosphorus-oxygen bonds, phosphorus-nitrogen bonds, and combinations thereof.

In some embodiments of Formula 1-3, NBM is an anthraquinoid moiety having a substituted 9,10-anthraquinone core structure. In some embodiments, the DNA-binding compounds are of Formula 4:

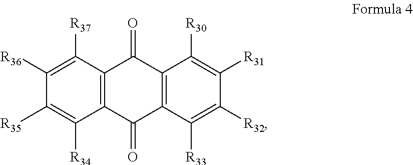

or a salt thereof, wherein:
R₃₀-R₃₇ are independently selected from H, halogen, hydroxy, an optionally substituted amino, optionally substituted alkyl, optionally substituted alkoxy, optionally substituted aryloxy, optionally substituted aryl, optionally substituted heteroaryl and L-BRM, wherein at least one of R₃₀-R₃₇ is -L-BRM, wherein BRM is a nucleic acid base-reactive moiety and L is a linker.

The present disclosure provides methods of detecting nucleic acids in a sample. A sample can be contacted with a nucleic acid detecting compounds to modify certain nucleic acids in the sample that are available for modification, e.g., free nucleic acid that is extracellular. In some embodiments, the method includes PCR-based detection of modified DNA from a sample. In some embodiments, the method includes PCR-based detection of unmodified DNA from a sample.

In some embodiments, a method of detecting nucleic acids in a sample includes:
a) contacting a sample that contains a nucleic acid with a compound of the present disclosure;
b) incubating the contacted sample under conditions sufficient for the compound to associate (e.g., covalently link) with a nucleic acid in the sample to produce a modified nucleic acid (e.g., nucleic acid complex) that prevents the nucleic acid of the modified nucleic acid from being amplified;
c) extracting nucleic acid from the sample;
d) purifying the extracted nucleic acid; and
e) detecting a signal of an amplified nucleic acid from the extracted nucleic acid.

In some embodiments, a kit for detecting nucleic acids in a sample is disclosed, the kit comprising:
a) a compound of the present disclosure; and
b) one or more components selected from a buffer, a nucleic acid standard, a DNA or RNA ladder, a detergent, a matrix, and an instruction sheet concerning use of the kit for detecting nucleic acids in a sample.

### BRIEF DESCRIPTION OF THE DRAWINGS

A detailed description of various aspects, features and embodiments is provided herein with reference to the accompanying drawings, which are briefly described below. The drawings are illustrative and are not necessarily drawn to scale. The drawings illustrate various aspects or features and may illustrate one or more embodiment(s) or example(s) in whole or in part. A reference numeral, letter, and/or symbol that are used in one drawing to refer to a particular element or feature may be used in another drawing to refer to a like element or feature.

FIG. 1 illustrates the use of the compounds of the present disclosure in viability PCR. Step 1. A sample of mixed live (solid line) and dead bacteria (dotted line) is treated with a compound of the disclosure. Step 2. The DNA is extracted and purified from the sample, amplified and detected via PCR.

FIGS. 2A-2C illustrate normalized qPCR curves from a viability PCR experiment in which live, and heat-inactivated E. coli are treated without an inhibitor (control), PMA (with UV light irradiation) or Compound 8 (without light activation). qPCR is performed using primers against a region of the uidA gene. The treatment of Compound 8 has no effect on amplification of DNA from live E. coli but causes a dramatic delay in amplification of DNA from heat-killed E. coli. On the contrary, in the absence of light, PMA does not have any effect on the amplification of DNA from heat-killed E. coli.

### DETAILED DESCRIPTION

### Definitions

The following definitions are set forth to illustrate and define the meaning and scope of the various terms used herein.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Compounds of this disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry, 5^{th} Ed., Ed.: Smith, M. B. and March, J., John Wiley & Sons, New York: 2001, the entire contents of which are hereby incorporated by reference.

The abbreviations used herein have their conventional meaning without the chemical and biological arts. The chemical structures and formulae set forth herein are constructed according to the standard rules of chemical valency known in the chemical arts.

The term “alkyl,” as used herein, by itself or as part of another group, refers to straight, branched chain or cyclic radicals having up to 50 carbons and 10 heteroatoms, unless the chain length or ring size is limited thereto, such as methyl, ethyl, propyl, cyclopropanyl, isopropyl, butyl, t-butyl, isobutyl, pentyl, hexyl, cyclohexyl, isohexyl, heptyl, 4,4-dimethylpentyl, octyl, 2,2,4-trimethylpentyl, nonyl, and decyl, among others.

The alkyl moieties may be substituted or contain other moieties including but not limited to carbonyl, ester, urea, urethane, sulfonamide, sulfone, or amide groups.

The term “alkylene” as used herein, by itself or as part of another group, refers to straight, branched chain or cyclic divalent radicals having up to 50 carbons, unless the chain length or ring size is limited thereto. Typical examples include methylene (—CH₂—), ethylene (—CH₂CH₂—), propylene, butylene, pentylene, hexylene, heptylene, octylene, nonylene, and decylene, among others.

The term “alkenyl,” as used herein, by itself or as part of another group, means a straight, branched chain or cyclic radical having 2-50 carbon atoms and one or more carbon-carbon double bonds, unless the chain length or ring size is limited thereto, such as ethenyl, 1-propenyl, 2-propenyl, 2-methyl-1-propenyl, 1-butenyl, and 2-butenyl, among others. The alkenyl chain may be 2 to 10 carbon atoms in length. Alternatively, the alkenyl chain may be 2 to 4 carbon atoms in length.

The term “alkenylene,” as used herein, by itself or as part of another group, means straight, branched chain or cyclic divalent radical having 2-50 carbon atoms, unless the chain length or ring size is limited thereto, said straight, branched chain or cyclic radical containing at least one carbon-carbon double bond. Typical examples include ethenylene (—CH═CH—), propenylene (—CH═CHCH₂— and —CH₂CH═CH—), n-butenylene, and 3-methyl-2-pentenylene, hexenylene, heptenylene, octenylene, nonenylene, and decenylene, among others.

The term “alkynyl,” as used herein, by itself or as part of another group, means a straight, branched chain or cyclic radical of 2-50 carbon atoms, unless the chain length or ring size is limited thereto, having at least one carbon-carbon triple bond between two of the carbon atoms in the chain, such as acetylenyl, 1-propynyl, and 2-propynyl, among others. The alkynyl chain may be 2 to 10 carbon atoms in length. Alternatively, the alkynyl chain may be from 2 to 4 carbon atoms in length.

The term “alkynylene” as used herein, by itself or as part of another group, means a straight, branched chain or cyclic divalent radical having 2-50 carbon atoms, unless the chain length or ring size is limited thereto, that contains at least one carbon-carbon triple bond. Typical examples include ethynylene (—C≡C—), propynylene (—C≡CCH₂— and —CH₂C≡C—), n-butynylene, 4-methyl-2-pentynylene, 1-butynylene, 2-butynylene, 3-butynylene, 4-butynylene, pentynylene, hexynylene, heptynylene, octynylene, nonynylene, and decynylene, among others.

The term “alkoxy,” as used herein, by itself or as part of another group, refers to any of the above radicals linked via an oxygen atom. Typical examples include methoxy, ethoxy, isopropyloxy, sec-butyloxy, n-butyloxy, t-butyloxy, n-pentyloxy, 2-methylbutyloxy, 3-methylbutyloxy, n-hexyloxy, and 2-ethylbutyloxy, among others. Alkoxy also may include polyethylene glycol (PEG) groups (—OCH₂CH₂O—) or alkyl moieties that contain more than one oxygen atom. The alkoxy moieties may be substituted or contain other moieties including but not limited to carbonyl, ester or amide groups.

The term “aryl,” as used herein, by itself or as part of another group, refers to an aryl or aromatic ring system containing 1 to 4 unsaturated rings (each ring containing 6 conjugated carbon atoms and no heteroatoms) that are optionally fused to each other or bonded to each other by carbon-carbon single bonds, that is optionally further substituted as described below. Examples of aryl ring systems include, but are not limited to, substituted or unsubstituted derivatives of phenyl, biphenyl, o-, m-, or p-terphenyl, 1-naphthyl, 2-naphthyl, 1-, 2-, or 9-anthryl, 1-, 2-, 3-, 4-, or 9-phenanthrenyl and 1-, 2- or 4-pyrenyl. Aryl substituents may include phenyl, substituted phenyl, naphthyl or substituted naphthyl. The aryl moieties may be substituted or contain other moieties including but not limited to carbonyl, ester, urea, urethane, sulfonamide, sulfone, or amide groups.

The term “arylene,” as used herein, by itself or as part of another ring, refers to an aryl or aromatic ring system containing 1 to 4 unsaturated rings (each ring containing 6 conjugated carbon atoms and no heteroatoms) that are optionally fused to each other or bonded to each other by carbon-carbon single bonds, that is optionally further substituted as described below. Examples of aryl ring systems include, but are not limited to, substituted or unsubstituted derivatives of phenylene, biphenylene, o-, m-, or p-terphenylene, 1-naphthylene, 2-naphthylene, 1-, 2-, or 9-anthrylene, 1-, 2-, 3-, 4-, or 9-phenanthrenylene and 1-, 2- or 4-pyrenylene. Arylene substituents may include phenylene, substituted phenylene, naphthylene or substituted naphthylene. The arylene moieties may be substituted or contain other moieties including but not limited to carbonyl, ester, urea, urethane, sulfonamide, sulfone, or amide groups.

The term “heteroaryl,” as used herein, by itself or as part of another group, refers to groups having 5 to 14 ring atoms; 6, 10 or 14 π electrons shared in a cyclic array; and containing carbon atoms and 1, 2, 3, or 4 oxygen, nitrogen or sulfur heteroatoms (where examples of heteroaryl groups are: thienyl, benzo[b]thienyl, naphtho[2,3-b]thienyl, thianthrenyl, furyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, carbazolyl, phenanthridinyl, acridinyl, perimidinyl, phenanthrolinyl, phenazinyl, isothiazolyl, phenothiazinyl, isoxazolyl, furazanyl, phenoxazinyl, and tetrazolyl groups). The heteroaryl moieties may be substituted or contain other moieties including but not limited to carbonyl, ester, urea, urethane, sulfonamide, sulfone, or amide groups.

The term “heteroarylene,” as used herein, by itself or as part of another ring, refers to groups having 5 to 14 ring atoms; 6, 10 or 14 π electrons shared in a cyclic array; and containing carbon atoms and 1, 2, 3, or 4 oxygen, nitrogen or sulfur heteroatoms (where examples of heteroaryl groups are: thienylene, benzo[b]thienylene, naphtho[2,3-b]thienylene, thianthrenylene, furylene, pyranylene, isobenzofuranylene, benzoxazolylene, chromenylene, xanthenylene, phenoxathiinylene, 2H-pyrrolylene, pyrrolylene, imidazolylene, pyrazolylene, pyridylene, pyrazinylene, pyrimidinylene, pyridazinylene, indolizinylene, isoindolylene, 3H-indolylene, indolylene, indazolylene, purinylene, 4H-quinolizinylene, isoquinolylene, quinolylene, phthalazinylene, naphthyridinylene, quinazolinylene, cinnolinylene, pteridinylene, carbazolylene, phenanthridinylene, acridinylene, perimidinylene, phenanthrolinylene, phenazinylene, isothiazolylene, phenothiazinylene, isoxazolylene, furazanylene, phenoxazinylene, and tetrazolylene rings). The heteroarylene moieties may be substituted or contain other moieties including but not limited to carbonyl, ester, urea, urethane, sulfonamide, sulfone, or amide groups.

Any aryl, arylene, heteroaryl or heteroarylene ring system is unsubstituted or optionally and independently substituted by any synthetically accessible and chemically stable combination of substituents, such as H, halogen, cyano-, sulfo-, alkali or ammonium salt of sulfo-, nitro, carboxy, alkyl, perfluoroalkyl, alkoxy, alkylthio, amino, monoalkylamino, dialkylamino or alkylamido-, the alkyl portions of which having 18 or fewer carbons.

The terms “halogen” or “halo,” as used herein, by itself or as part of another group, refers to chlorine, bromine, fluorine or iodine.

The terms “amino” or “amine” include NH₂, “monoalkylamine” or “monoalkylamino,” and “dialkylamine” or “dialkylamino”. The terms “monoalkylamine” and “monoalkylamino,” “dialkylamine” and “dialkylamino as used herein, by itself or as part of another group, refers to the group NH₂ where one hydrogen has been replaced by an alkyl group, as defined above.

The terms “dialkylamine” and “dialkylamino,” as used herein, by itself or as part of another group, refer to the group NH₂ where both hydrogens have been replaced by alkyl groups, as defined above.

The term “hydroxyalkyl,” as used herein, by itself or as part of another group, refers to an alkyl group where one or more hydrogens thereof are substituted by one or more hydroxyl moieties.

The term “haloalkyl,” as used herein, by itself or as part of another group, refers to an alkyl group where one or more hydrogens thereof are substituted by one or more halo moieties. Typical examples include chloromethyl, fluoromethyl, difluoromethyl, trifluoromethyl, trichloroethyl, trifluoroethyl, fluoropropyl, and bromobutyl, among others.

The term “haloalkenyl,” as used herein, by itself or as part of another group, refers to an alkenyl group where one or more hydrogens thereof are substituted by one or more halo moieties.

The term “haloalkynyl,” as used herein, by itself or as part of another group, refers to an alkynyl group where one or more hydrogens thereof are substituted by one or more halo moieties.

The term “carboxyalkyl,” as used herein, by itself or as part of another group, refers to an alkyl group where one or more hydrogens thereof are substituted by one or more carboxylic acid moieties.

The term “heteroatom” as used herein, by itself or as part of another group, means an oxygen atom (“O”), a sulfur atom (“S”) or a nitrogen atom (“N”). It will be recognized that when the heteroatom is nitrogen, it may form an NR₁R₂ moiety, where R₁ and R₂ are, independently from one another, hydrogen or alkyl, or together with the nitrogen to which they are bound, form a saturated or unsaturated 5-, 6-, or 7-membered ring.

The term “nucleic acid” refers to double-stranded DNA (dsDNA), single-stranded DNA (ssDNA), double-stranded RNA (dsRNA), single-stranded RNA (ssRNA), and/or derivatives thereof. A nucleic acid may be natural or synthetic.

The term “counterion (e.g., W)” refers to a biologically compatible ion that is stable and synthetically accessible. Examples of counterions include, among others, chloride, bromide, iodide, sulfate, phosphate, perchlorate, tetrafluoroborate, nitrate, trifluoroacetate and anions of aromatic or aliphatic carboxylic acids, etc.

The term “linker” refers to a moiety used to connect a BRM to a NBM together. The linker which may be charged or neutral, is a flexible chain containing 1 to 100 non-hydrogen atoms. It may be an alkyl, an aryl or a heteroaryl. The linker may contain at least one of heteroatoms N, O, or S, e.g. polyethylene glycol (PEG), which may be the same or different.

The term “heteroatom” means one or more of oxygen, sulfur, nitrogen, phosphorus, or silicon (including, any oxidized form of nitrogen, sulfur, phosphorus, or silicon; the quaternized form of any basic nitrogen; or a substituted nitrogen of a heterocyclic ring).

The term “polyamine,” as used herein, refers to a linear linker having a backbone of 4 or more consecutive atoms, comprising one or more divalent linking moieties independently selected from —NH— and —NMe-.

The term “polyammonium,” as used herein refers to a linear linker having a backbone of 4 or more consecutive atoms, comprising one or more divalent linking moieties independently selected from —⁺NH₂— and —⁺NMe₂-.

### Compounds

The present disclosure is directed to compounds which are useful for the detection, discrimination and quantification of biological targets and events. Embodiments of the present disclosure provide nucleic acid detecting compounds having one nucleic acid binding moiety (NBM) moiety to provide for binding of the compound to nucleic acids, and at least one nucleic acid base-reactive moiety (BRM) to provide for formation of a covalent linkage to the nucleic acid. These compounds are found to be used as nucleic acid probes, particularly for use in assays for the selective detection of certain nucleic acids, such as DNA. In some embodiments, the nucleic acid detecting compounds are used in conjunction with a viability PCR assay to distinguish DNA from live versus dead cells.

The term “nucleic acid binding moiety” or “NBM,” as used herein, refers to a moiety capable of non-covalently binding to a nucleic acid. In some embodiments, the binding is non-sequence specific, such that the NBM binds generally to nucleic acids but is not capable of specifically binding to any one polynucleotide sequence. In some embodiments, the binding is sequence specific, such that the NBM binds specifically to a particular polynucleotide sequence. In some embodiments, the NBM binds via intercalation (insertion) between the planar nucleobases of a nucleic acid. In some embodiments, the NBM is moiety including a planar optionally substituted polycyclic aryl or heteroaryl moiety that binds nucleic acid. In some embodiments, the NBM binds via non-covalent binding to a structural element of the nucleic acid double helix, such as a minor groove binder.

The term “nucleic acid base-reactive moiety” or “BRM,” as used herein, refers to a moiety that includes a reactive functional group capable of forming a covalent bond with a complementary group of a nucleotide of a nucleic acid. The formation of the covalent bond linkage may be proximity induced, e.g., promoted by binding of the NBM to the nucleic acid. In some embodiments, the reactive functional group is capable of reaction with a compatible group of the nucleic acid without activation. The compatible group of the nucleic acid can be part of the nucleobase, the sugar moiety or the phosphodiester backbone. A variety of nucleic acid labelling methods and reagents are known to the skilled artisan and may be adapted for use in the compounds of this disclosure. For example, a 3′-terminal or 5′-terminal (hydroxyl or phosphate terminal), 2′-hydroxyl group, or nucleobase group, of a nucleic acid can be modified by reaction with an electrophilic BRM of the compounds of this disclosure. In some embodiments, the reactive functional group is masked and becomes capable of reaction with a compatible group of the nucleic acid after activation, e.g., activation with a stimulus such as irradiation with light, or exposure to a stimulus in situ, such as a low pH condition. In some embodiments, the reactive functional group is electrophilic and forms a covalent bond with a complementary nucleophilic group of a nucleic acid substitution.

In one embodiment, the DNA-binding compounds may be described by Formula 1:

[BRM1-L1]ₘ-NBM-[L2-BRM2]ₙ Formula 1

or a salt thereof, wherein:
BRM1 and BRM2 are independently a nucleic acid base-reactive moiety;
NBM is a nucleic acid binding moiety;
L1 and L2 are independently a linker; and
m and n are independently an integer from 0 to 4, wherein the sum of m and n is from 1 to 4.

In one aspect, the DNA-binding compounds of the disclosure may be described by the following formula:

{[BRM1-L1]ₘ-NBM-[L2-BRM2]ₙ}W

In this embodiment, wherein BRM1 and BRM2 are independently a nucleic acid base-reactive moiety; NBM is a nucleic acid binding moiety; L1 and L2 are independently a linker; m and n are independently an integer from 0 to 4, provided that the total of m and n is from 1 to 4; and W is none or a physiologically acceptable counter ion.

In some embodiments, m and n are independently 1, 2, 3, or 4. In some embodiments, the sum of m and n is 1. In some embodiments, the sum of m and n is 2. In some embodiments, m and n are each 1. In some embodiments, m and n are independently 1 or 2. In some embodiments, the sum of m and n is 3. In some embodiments, the sum of m and n is 4.

In some embodiments, BRM1 and BRM2 are independently selected from a 2-chloroethylamino moiety, a 2-bromoethylamino moiety, a 2-iodoethylamino moiety, an aziridinyl, an enediynyl, a psoralen, an azidoaryl, a quinomethide-generating moiety, a N-acetoxy-N-2-acetylaminofluorenyl, a 4-acetoxy-3-fluoromethyl-phenyl moiety, and a platinum complex.

In some embodiments, BRM1 and BRM2 are independently selected from a 2-chloroethylamino moiety, a 2-bromoethylamino moiety, a 2-iodoethylamino moiety, an aziridinyl, an enediynyl, a psoralen, a quinomethide-generating moiety, a N-acetoxy-N-2-acetylaminofluorenyl, a 4-acetoxy-3-fluoromethyl-phenyl moiety, and a platinum complex.

In some embodiments, BRM1 and BRM2 are each independently selected from a Cl—C(R₁R₂)—C—(R₃R₄)—N(R)—, Br—C(R₁R₂)—C—(R₃R₄)—N(R₅)—, and I—C(R₁R₂)—C—(R₃R₄)—N(R₅)—, wherein R₁ to R₅ are independently selected from optionally substituted alkyl, aryl, and heteroaryl.

In some embodiments, BRM1 and BRM2 are each independently selected from:

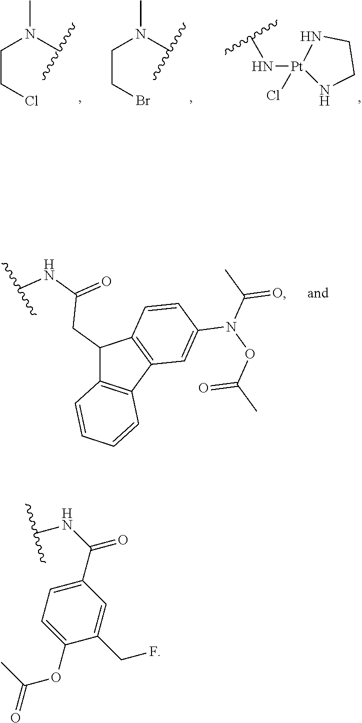

In some embodiments, NBM is an intercalator. or a minor groove binding moiety.

In some embodiments, NBM is selected from a phenanthridinum moiety, a proflavine, an anthraquinoid moiety, an actinomycin D moiety, an acridine, a netropsin moiety, a distamycin moiety, a Hoechst moiety, a beranil moiety, and a DAPI moiety.

In some embodiments, NBM is selected from a phenanthridinum moiety, an anthraquinoid moiety, a netropsin moiety, a Hoechst moiety, and a beranil moiety. In some embodiments, NBM is an anthraquinoid moiety.

In some embodiments, NBM is an anthraquinoid moiety; m and n are each 1; and BRM1 and BRM2 are each a 2-chloroethylamino moiety.

In some embodiments, L1 and L2 are independently a linker comprising 1-100 non-hydrogen atoms selected from C, N, O, P, and S, further comprising a plurality of single, double, triple or aromatic carbon-carbon bonds, carbon-nitrogen bonds, nitrogen-nitrogen bonds, carbon-oxygen bonds, carbon-sulfur bonds, phosphorus-oxygen bonds, phosphorus-nitrogen bonds, and combinations thereof.

In some embodiments, L1 and L2 are each independently selected from an alkyl, an aryl, a heteroaryl, a arylene, an ether and a thioether, wherein the alkyl, the aryl, the heteroaryl, the arylene, the ether and the thioether are optionally substituted.

In some embodiments, L1 and L2 are independently a linear linker having a backbone of 4 or more consecutive atoms, comprising one or more divalent linking moieties independently selected from —C₁₋₂₀-alkylene-, —NHCO—, —CONH—, —NH—, —⁺NH₂—, —NMe-, —⁺NMe₂-, —NHCONH—, —NHCSNH—, —O(CH₂)ₚ—, —(OCH₂CH₂)ₚ—, —NHSO₂—, —SO₂NH—, —CO—, —SO₂—, —O—, and —S—, wherein each p is independently 1 to 10.

In some embodiments, L1 and L2 are independently a linear linker having a backbone of 4 to 24 consecutive atoms, comprising 1 to 8 divalent linking moieties independently selected from —C₁₋₆-alkylene-, —NHCO—, —CONH—, —NH—, —⁺NH₂—, —NMe-, —⁺NMe₂-, —O(CH₂)ₚ—, —(OCH₂CH₂)ₚ—, —CO—, and —O—, wherein each p is independently 1 to 10.

In some embodiments, wherein L1 and L2 are independently a linear linker having a backbone of 8 to 20 consecutive atoms, comprising 1 to 8 divalent linking moieties independently selected from —NHCO—, —CONH—, —NH—, —NMe-, —⁺NH₂—, and —⁺NMe₂.

In some embodiments, L1 and L2 are independently selected from:

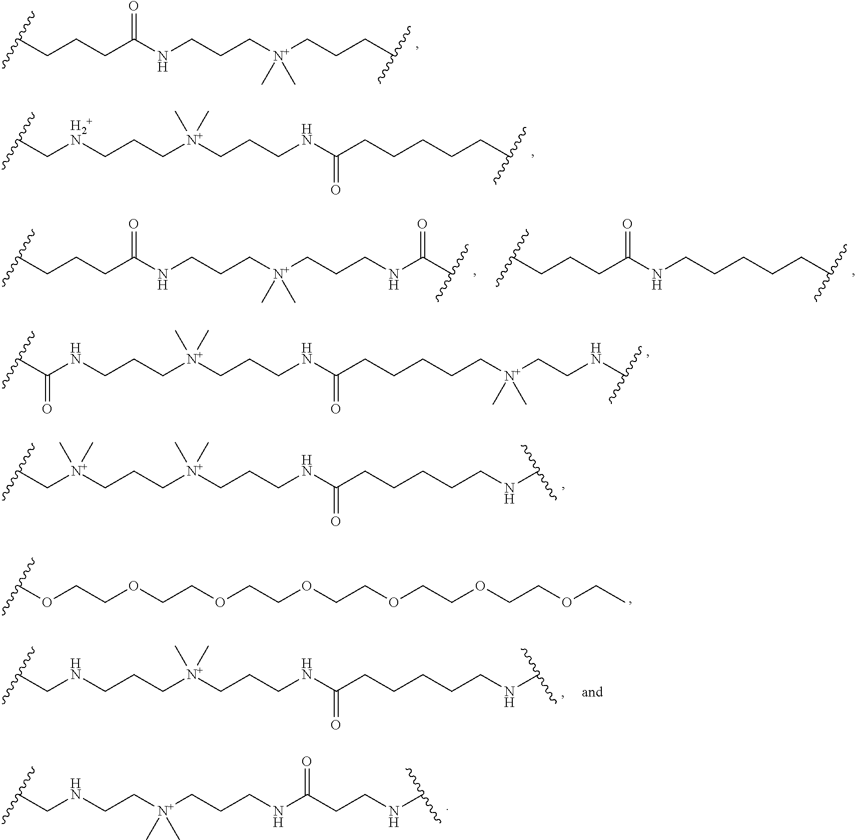

In some embodiments, L1 and L2 are each independently a polyethylene glycol (PEG) linking moiety. In some embodiments, L1 and L2 are each independently comprise repeating ethylene glycol moieties (e.g., —CH₂CH₂O— or —OCH₂CH₂—).

In some embodiments, the polyethylene glycol (PEG) linking moiety comprises 1 to 20 monomers of ethylene glycol (e.g., 1 to 8, 2 to 8, or 4 to 8 monomers).

In some embodiments, L1 and L2 are independently a polyamine or a polyammonium. In some embodiments, L1 and L2 independently comprise a polyamine linking moiety (e.g., —[N⁺(R)₂-C₍₁₋₆₎alkyl]ₙ- wherein R is H or methyl and n is 1-6, such as 1, 2, or 3) or a polyammonium linking moiety (e.g., —[NR—C₍₁₋₆₎alkyl]ₙ- wherein R is H or methyl and n is 1-6, such as 1, 2, or 3).

In some embodiments, L1 and L2 independently comprise an oxo (e.g., —O—) and/or arylene.

In some embodiments, L1 and L2 are independently represented by Formula 1′:

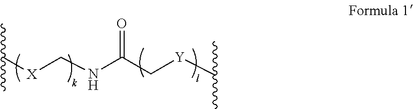

wherein:
each X and each Y are independently selected from optionally substituted alkyl (e.g., C1-6alkyl, ether, amino group, amide group, oxo, ammonium, aryl, and heteroaryl; and
k and l are independently an integer from 0 to 10.

In some embodiments, X and Y are selected from optionally substituted alkyl, amino group, amide group, and ammonium; and k and l are independently an integer from 1 to 6.

Specific examples of L1 and L2 include substituted or unsubstituted polyamine, polymethylene, aryl, heteroaryl, arylene, alkylarylene or arylenealkyl. In yet another embodiment, L1 and L2 are independently a thioether linkage. In yet another embodiment, L1 and L2 are independently a PEG linkage.

Specific examples of aryl ring systems include substituted or unsubstituted phenyl, biphenyl, naphthyl, anthryl, phenanthrenyl or pyrenyl.

Specific examples of heteroaryl ring systems include substituted or unsubstituted thienyl, benzothienyl, naphthothienyl, furyl, pyranyl, isobenzofuranyl, benzoxazolyl, chromenyl, xanthenyl, phenoxathiinyl, pyrrolyl, imidazolyl, pyrazolyl, pyridyl, pyrazinyl, pyrimidinyl, indolyl, purinyl, quinolyl, phthalazinyl, naphthyridinyl, quinazolinyl, cinnolinyl, carbazolyl, phenanthridinyl, acridinyl, phenazinyl, thiazolyl, oxazolyl, furazanyl, phenoxazinyl, or tetrazolyl.

Specific examples of arylene ring systems include substituted or unsubstituted phenylene, biphenylene, naphthylene, anthrylene, phenanthrenylene or pyrenylene.

Specific examples of heteroarylene ring systems include substituted or unsubstituted thienylene, benzothienylene, naphthothienylene, furylene, pyranylene, isobenzofuranylene, benzoxazolylene, chromenylene, xanthenylene, phenoxathiinylene, pyrrolylene, imidazolylene, pyrazolylene, pyridylene, pyrazinylene, pyrimidinylene, indolylene, purinylene, quinolylene, phthalazinylene, naphthyridinylene, quinazolinylene, cinnolinylene, carbazolylene, phenanthridinylene, acridinylene, phenazinylene, thiazolylene, oxazolylene, furazanylene, phenoxazinylene, or tetrazolylene.

In some embodiments, the compound is a salt comprising one or more counterion (W). Specific examples of W are physiological counter ions, e.g., fluoride, chloride, bromide, iodide, sulfate, sulfite, bisulfite, thiosulfate, phosphate, hydrogen phosphate, dihydrogen phosphate, thiophosphate, phosphite, nitrate, nitrite, borate, tetrafluoroborate, chlorate, chlorite, hypochlorite, perchlorate, carbonate, bicarbonate, formate, oxalate, acetate, trifluoroacetate, mesylate, tosylate, triflate, or thiocyanate, etc.

In another aspect, the compounds may be described by Formula 2:

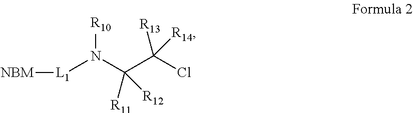

or a salt thereof.

In this embodiment, NBM is selected from an intercalator and a minor groove binding moiety; R₁₀-R₁₄ are independently selected from H, an alkyl, an alkoxy, an aryloxy, an aryl, and a heteroaryl; and L1 is a linker comprising 1-100 non-hydrogen atoms selected from C, N, O, P, and S, further comprising a plurality of single, double, triple or aromatic carbon-carbon bonds, carbon-nitrogen bonds, nitrogen-nitrogen bonds, carbon-oxygen bonds, carbon-sulfur bonds, phosphorus-oxygen bonds, phosphorus-nitrogen bonds, and combinations thereof.

In some embodiments, NBM is selected from a phenanthridinum moiety, a proflavine, an anthraquinoid moiety, an actinomycin D moiety, an acridine, a netropsin moiety, a distamycin moiety, a Hoechst moiety, a beranil moiety, and a DAPI moiety; and L1 is represented by Formula 1′:

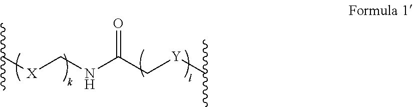

wherein:
each X and each Y are independently selected from optionally substituted alkyl, amino group, and ammonium, and
k and l are independently an integer from 1 to 6.

In some embodiments, NBM is selected from a phenanthridinum moiety, an anthraquinoid moiety, a netropsin moiety, a Hoechst moiety, and a beranil moiety.

In some embodiments, R₁₀-R₁₄ are independently selected from H and an alkyl.

In some embodiments, the compound is selected from the compounds of Table 1.

TABLE 1
Selected embodiments of the compounds of the disclosure.

| Cpd # | Structure |
|---|---|
| 15 | 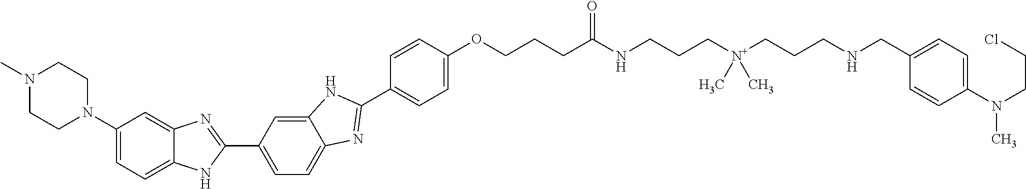 [SMILES: CN1CCN(c2ccc3[nH]c(-c4ccc5nc(-c6ccc(OCCCC(=O)NCCC[N+](C)(C)CCCNCc7ccc(N(C)CCCl)cc7)cc6)[nH]c5c4)nc3c2)CC1] |
| 18 | 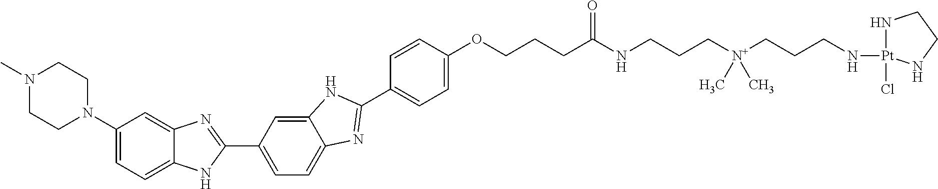 |
| 20 | 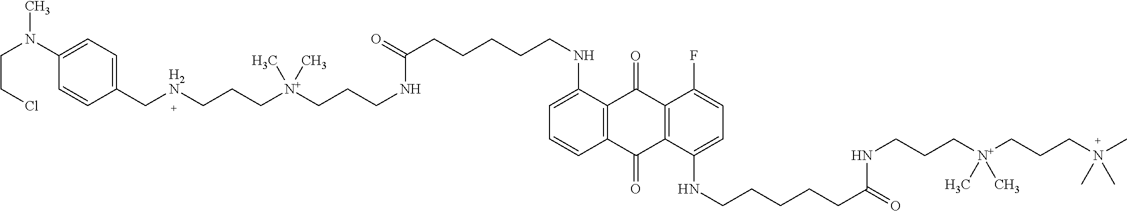 |
| 21 | 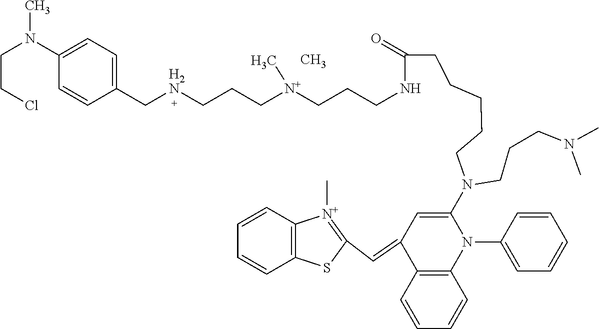 |
| 22 | 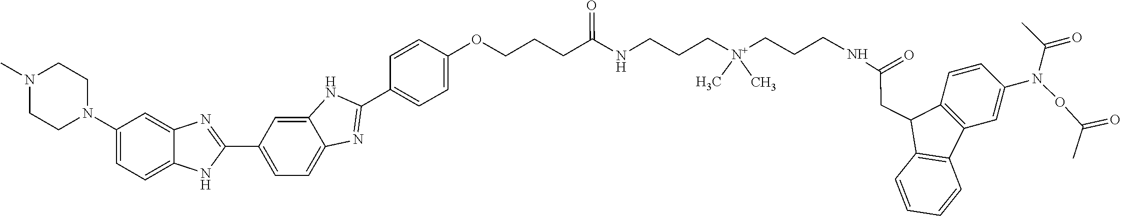 [SMILES: CC(=O)ON(C(C)=O)c1ccc2c(c1)-c1ccccc1C2CC(=O)NCCC[N+](C)(C)CCCNC(=O)CCCOc1ccc(-c2nc3ccc(-c4nc5cc(N6CCN(C)CC6)ccc5[nH]4)cc3[nH]2)cc1] |
| 23 | 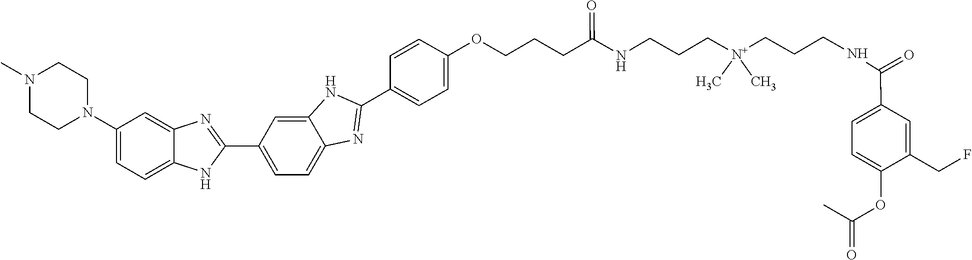 [SMILES: CC(=O)Oc1ccc(C(=O)NCCC[N+](C)(C)CCCNC(=O)CCCOc2ccc(-c3nc4ccc(-c5nc6cc(N7CCN(C)CC7)ccc6[nH]5)cc4[nH]3)cc2)cc1CF] |
| 26 | 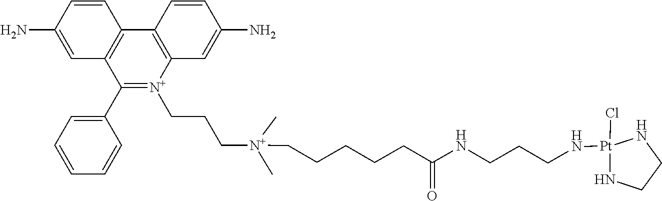 |
| 27 | 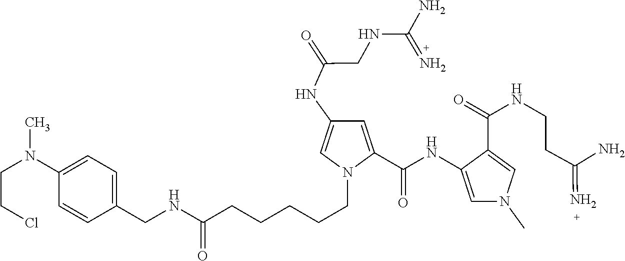 [SMILES: CN(CCCl)c1ccc(CNC(=O)CCCCCn2cc(NC(=O)CNC(=N)N)cc2C(=O)Nc2cn(C)cc2C(=O)NCCC(=N)N)cc1] |
| 31 | 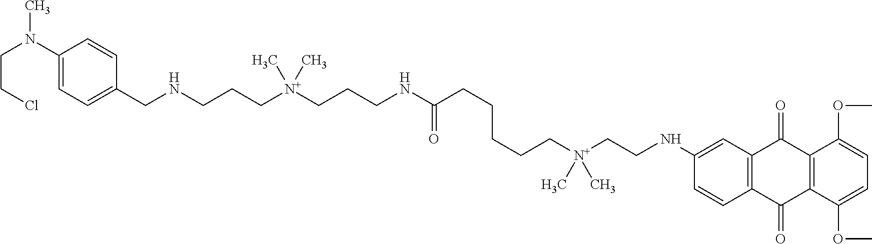 [SMILES: COc1ccc(OC)c2c1C(=O)c1ccc(NCC[N+](C)(C)CCCCCC(=O)NCCC[N+](C)(C)CCCNCc3ccc(N(C)CCCl)cc3)cc1C2=O] |
| 32 | 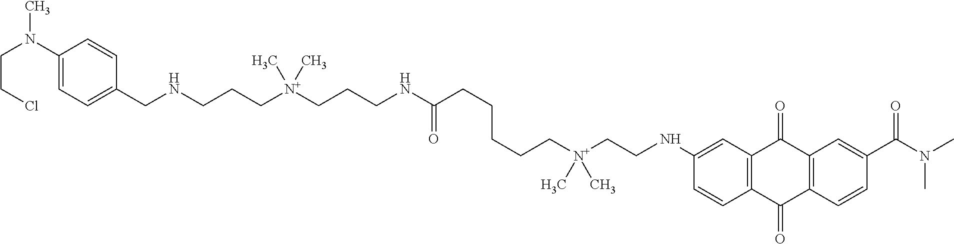 [SMILES: CN(C)C(=O)c1ccc2c(c1)C(=O)c1cc(NCC[N+](C)(C)CCCCCC(=O)NCCC[N+](C)(C)CCCNCc3ccc(N(C)CCCl)cc3)ccc1C2=O] |
| 33 | 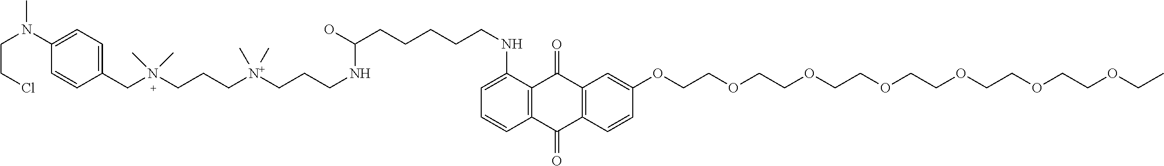 |

In another aspect, the compounds may be described by Formula 3:

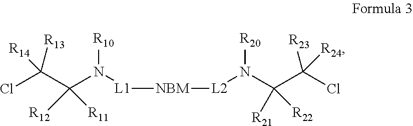

or a salt thereof.

In this embodiment, NBM is selected from an intercalator and a minor groove binding moiety; R₁₀-R₁₄ and R₂₀-R₂₄ are independently selected from H, an alkyl, an alkoxy, an aryloxy, an aryl and a heteroaryl; and L1 and L2 are each independently a covalent linkage having 1-100 non-hydrogen atoms selected from C, N, O, P, and S, further comprising a plurality of single, double, triple or aromatic carbon-carbon bonds, carbon-nitrogen bonds, nitrogen-nitrogen bonds, carbon-oxygen bonds, carbon-sulfur bonds, phosphorus-oxygen bonds, phosphorus-nitrogen bonds, and combinations thereof.

In some embodiments, NBM is selected from a phenanthridinum moiety, a proflavine, an anthraquinoid moiety, an actinomycin D moiety, an acridine, a netropsin moiety, a distamycin moiety, a Hoechst moiety, a beranil moiety, and a DAPI moiety; L1 and L2 are each independently represented by Formula 1′

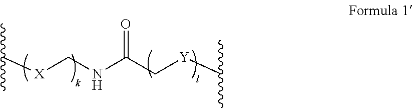

wherein:
each X and each Y are independently selected from optionally substituted alkyl, amino group, and ammonium; and
k and l are independently an integer from 1 to 6.

In some embodiments, NBM is an anthraquinoid moiety.

In some embodiments, R₁₀-R₁₄ and R₂₀-R₂₄ are independently selected from H and an alkyl.

In some embodiments, the compound is selected from the compounds of Table 2.

TABLE 2
Selected embodiments of the compounds of the disclosure.

| Cpd | |
|---|---|
| # | Structure |
| 8 | 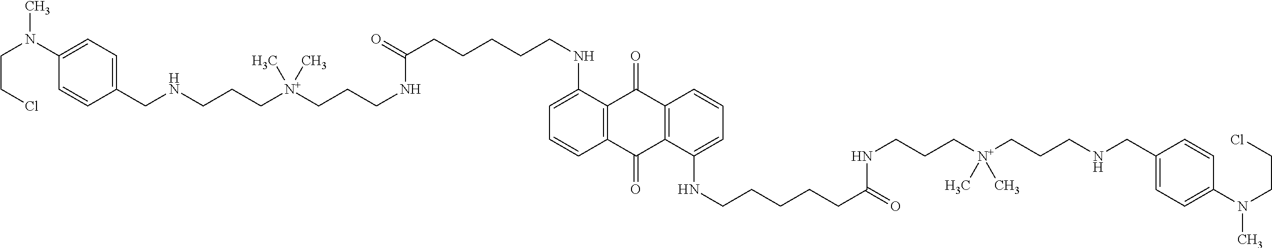 [SMILES: CN(CCCl)c1ccc(CNCCC[N+](C)(C)CCCNC(=O)CCCCCNc2cccc3c2C(=O)c2cccc(NCCCCCC(=O)NCCC[N+](C)(C)CCCNCc4ccc(N(C)CCCl)cc4)c2C3=O)cc1] |
| 11 | 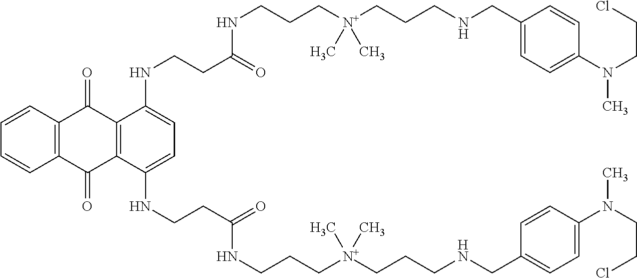 [SMILES: CN(CCCl)c1ccc(CNCCC[N+](C)(C)CCCNC(=O)CCNc2ccc(NCCC(=O)NCCC[N+](C)(C)CCCNCc3ccc(N(C)CCCl)cc3)c3c2C(=O)c2ccccc2C3=O)cc1] |
| 14 | 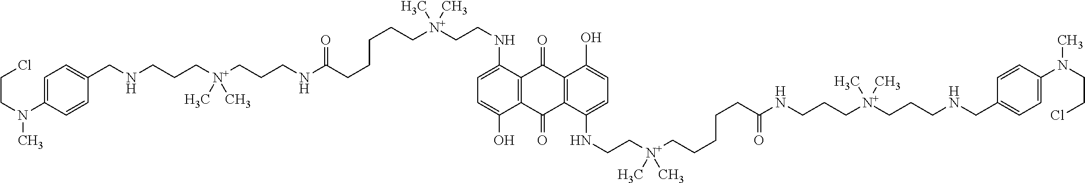 [SMILES: CN(CCCl)c1ccc(CNCCC[N+](C)(C)CCCNC(=O)CCCCC[N+](C)(C)CCNc2ccc(O)c3c2C(=O)c2c(O)ccc(NCC[N+](C)(C)CCCCCC(=O)NCCC[N+](C)(C)CCCNCc4ccc(N(C)CCCl)cc4)c2C3=O)cc1] |
| 28 | 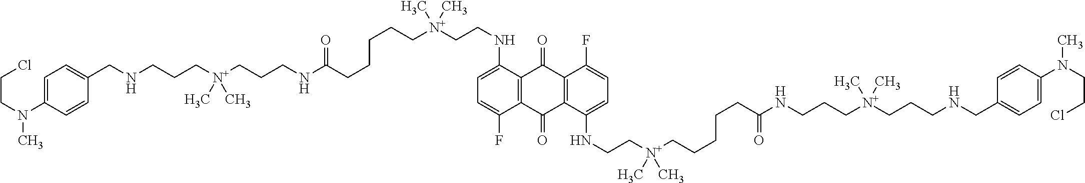 [SMILES: CN(CCCl)c1ccc(CNCCC[N+](C)(C)CCCNC(=O)CCCCC[N+](C)(C)CCNc2ccc(F)c3c2C(=O)c2c(F)ccc(NCC[N+](C)(C)CCCCCC(=O)NCCC[N+](C)(C)CCCNCc4ccc(N(C)CCCl)cc4)c2C3=O)cc1] |
| 29 | 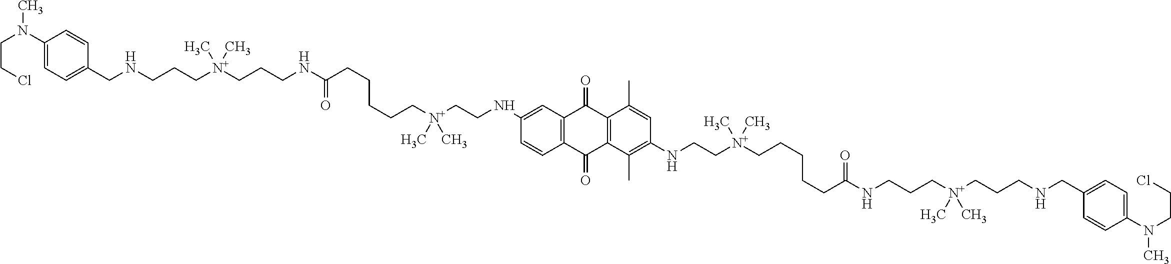 [SMILES: Cc1cc(NCC[N+](C)(C)CCCCCC(=O)NCCC[N+](C)(C)CCCNCc2ccc(N(C)CCCl)cc2)c(C)c2c1C(=O)c1cc(NCC[N+](C)(C)CCCCCC(=O)NCCC[N+](C)(C)CCCNCc3ccc(N(C)CCCl)cc3)ccc1C2=O] |
| 30 | 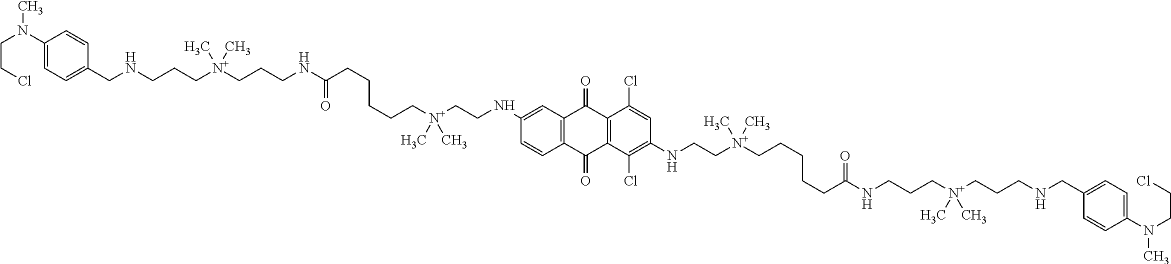 [SMILES: CN(CCCl)c1ccc(CNCCC[N+](C)(C)CCCNC(=O)CCCCC[N+](C)(C)CCNc2ccc3c(c2)C(=O)c2c(Cl)cc(NCC[N+](C)(C)CCCCCC(=O)NCCC[N+](C)(C)CCCNCc4ccc(N(C)CCCl)cc4)c(Cl)c2C3=O)cc1] |

In another aspect, the compounds may be described by Formula 4:

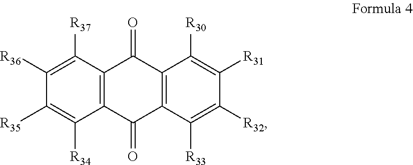

or a salt thereof.

In this embodiment, R₃₀-R₃₇ are independently selected from H, halogen, hydroxy, an optionally substituted amino, optionally substituted alkyl, optionally substituted alkoxy, optionally substituted aryloxy, optionally substituted aryl, optionally substituted heteroaryl and L-BRM, wherein at least one of R₃₀-R₃₇ is a L-BRM, wherein BRM is a nucleic acid base-reactive moiety and L is a linker.

In some embodiments, BRM is selected from a 2-chloroethylamino moiety, a 2-bromoethylamino moiety, a 2-iodoethylamino moiety, an aziridinyl, an enediynyl, a psoralen, an azidoaryl, a quinomethide-generating moiety, a N-acetoxy-N-2-acetylaminofluorenyl, and a platinum complex.

In some embodiments, BRM is selected from Cl—C(R₁R₂)—C—(R₃R₄)—N(R₅R₆), Br—C(R₁R₂)—C—(R₃R₄)—N(R₅R₆), or I—C(R₁R₂)—C—(R₃R₄)—N(R₅R₆) wherein R₁ to R₅ are each independently selected from optionally substituted alkyl, aryl, and heteroaryl.

In some embodiments, R₃₀ and R₃₃ are independently L-BRM; R₃₁, R₃₂ and R₃₄-R₃₇ are independently selected from H, bromo, chloro, hydroxy, amino, and an alkyl.

In some embodiments, R₃₀ and R₃₄ are L-BRM; R₃₁ to R₃₃ and R₃₅-R₃₇ are independently selected from H, bromo, chloro and an alkyl.

In some embodiments, R₃₁ and R₃₅ are each L-BRM; R₃₀, R₃₂ to R₃₄, R₃₆ and R₃₇ are independently selected from H, bromo, chloro or an alkyl.

In some embodiments, L1 and L2 are independently a linear linker having a backbone of 4 or more consecutive atoms, comprising one or more divalent linking moieties independently selected from —C₁₋₂₀-alkylene-, —NHCO—, —CONH—, —NH—, —⁺NH₂—, —NMe-, —⁺NMe₂-, —NHCONH—, —NHCSNH—, —O(CH₂)ₚ—, —(OCH₂CH₂)ₚ—, —NHSO₂—, —SO₂NH—, —CO—, —SO₂—, —O—, and —S—, wherein each p is independently 1 to 10.

In some embodiments, L1 and L2 are independently a linear linker having a backbone of 4 to 24 consecutive atoms, comprising 1 to 8 divalent linking moieties independently selected from —C₁₋₆-alkylene-, —NHCO—, —CONH—, —NH—, —⁺NH₂—, —NMe-, —⁺NMe₂-, —O(CH₂)ₚ—, —(OCH₂CH₂)ₚ—, —CO—, and —O—.

In some embodiments, L1 and L2 are independently a linear linker having a backbone of 8 to 20 consecutive atoms, comprising 1 to 8 divalent linking moieties independently selected from —NHCO—, —CONH—, —NH—, —NMe-, —NH₂—, and —⁺NMe₂.

In some embodiments, the compound is selected from the compounds of Table 3.

TABLE 3
Selected embodiments of the compounds of the disclosure.

| Cpd # | Structure |
|---|---|
| 8 | 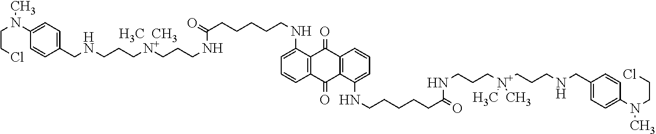 [SMILES: CN(CCCl)c1ccc(CNCCC[N+](C)(C)CCCNC(=O)CCCCCNc2cccc3c2C(=O)c2cccc(NCCCCCC(=O)NCCC[N+](C)(C)CCCNCc4ccc(N(C)CCCl)cc4)c2C3=O)cc1] |
| 11 | 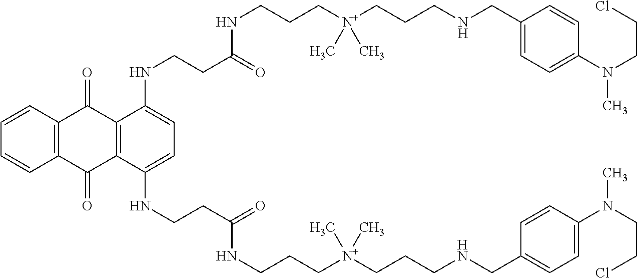 [SMILES: CN(CCCl)c1ccc(CNCCC[N+](C)(C)CCCNC(=O)CCNc2ccc(NCCC(=O)NCCC[N+](C)(C)CCCNCc3ccc(N(C)CCCl)cc3)c3c2C(=O)c2ccccc2C3=O)cc1] |
| 14 | 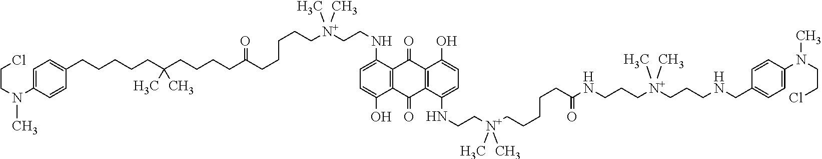 [SMILES: CN(CCCl)c1ccc(CCCCCC(C)(C)CCCCC(=O)CCCCC[N+](C)(C)CCNc2ccc(O)c3c2C(=O)c2c(O)ccc(NCC[N+](C)(C)CCCCCC(=O)NCCC[N+](C)(C)CCCNCc4ccc(N(C)CCCl)cc4)c2C3=O)cc1] |
| 20 | 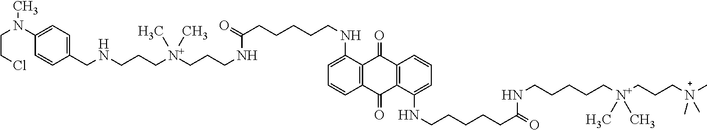 [SMILES: CN(CCCl)c1ccc(CNCCC[N+](C)(C)CCCNC(=O)CCCCCNc2cccc3c2C(=O)c2cccc(NCCCCCC(=O)NCCCCC[N+](C)(C)CCC[N+](C)(C)C)c2C3=O)cc1] |
| 28 | 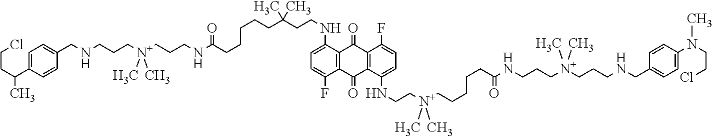 [SMILES: CC(CCCl)c1ccc(CNCCC[N+](C)(C)CCCNC(=O)CCCCCC(C)(C)CCNc2ccc(F)c3c2C(=O)c2c(F)ccc(NCC[N+](C)(C)CCCCCC(=O)NCCC[N+](C)(C)CCCNCc4ccc(N(C)CCCl)cc4)c2C3=O)cc1] |
| 29 | 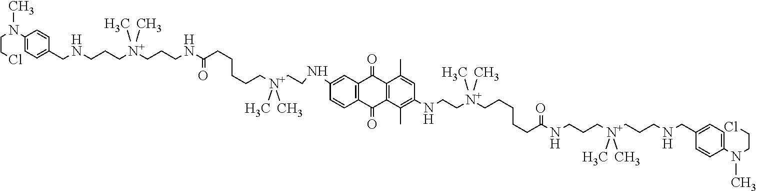 [SMILES: Cc1cc(NCC[N+](C)(C)CCCCCC(=O)NCCC[N+](C)(C)CCCNCc2ccc(N(C)CCCl)cc2)c(C)c2c1C(=O)c1cc(NCC[N+](C)(C)CCCCCC(=O)NCCC[N+](C)(C)CCCNCc3ccc(N(C)CCCl)cc3)ccc1C2=O] |
| 30 | 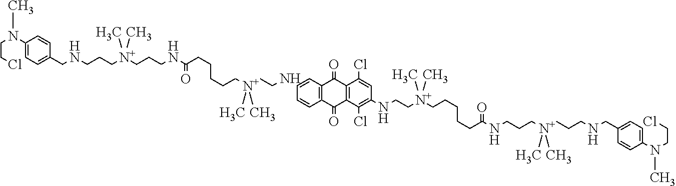 [SMILES: CN(CCCl)c1ccc(CNCCC[N+](C)(C)CCCNC(=O)CCCCC[N+](C)(C)CCNc2ccc3c(c2)C(=O)c2c(Cl)cc(NCC[N+](C)(C)CCCCCC(=O)NCCC[N+](C)(C)CCCNCc4ccc(N(C)CCCl)cc4)c(Cl)c2C3=O)cc1] |
| 31 | 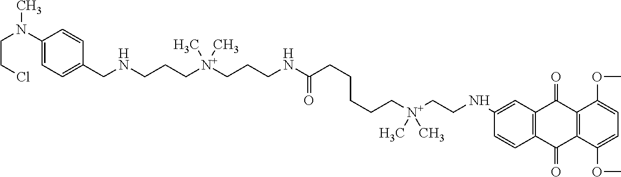 [SMILES: COc1ccc(OC)c2c1C(=O)c1ccc(NCC[N+](C)(C)CCCCCC(=O)NCCC[N+](C)(C)CCCNCc3ccc(N(C)CCCl)cc3)cc1C2=O] |
| 32 | 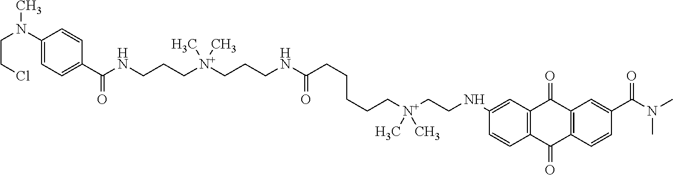 [SMILES: CN(C)C(=O)c1ccc2c(c1)C(=O)c1cc(NCC[N+](C)(C)CCCCCC(=O)NCCC[N+](C)(C)CCCNC(=O)c3ccc(N(C)CCCl)cc3)ccc1C2=O] |
| 33 | 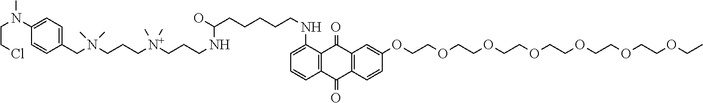 |

In some embodiments, the compound of Formula 1 is disclosed, wherein m and n are each 2; NBM is a beranil moiety; and L1 and L2 are an alkyl optionally substituted with 1 to 4 substituents selected from amino group, amide group, and ammonium. In some embodiments, BRM is selected from a 2-chloroethylamino moiety, 2-bromoethylamino moiety, 2-iodoethylamino moiety, an aziridinyl, an enediynyl, a psoralen, an azidoaryl, a quinomethide-generating moiety, a N-acetoxy-N-2-acetylaminofluorenyl, and a platinum complex. In some embodiments, BRM is selected from a 2-chloroethylamino moiety, 2-bromoethylamino moiety, and a platinum complex.

In some embodiments, the compound is selected from the compounds of Table 4.

TABLE 4
Selected embodiments of the compounds of the disclosure.

| Cpd # | Structure |
|---|---|
| 24 | 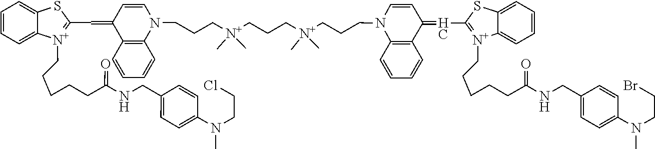 [SMILES: CN(CCCl)c1ccc(CNC(=O)CCCCC[n+]2c(C=C3C=CN(CCC[N+](C)(C)CCC[N+](C)(C)CCCN4C=CC(=Cc5sc6ccccc6[n+]5CCCCCC(=O)NCc5ccc(N(C)CCBr)cc5)c5ccccc54)c4ccccc43)sc3ccccc32)cc1] |
| 25 | 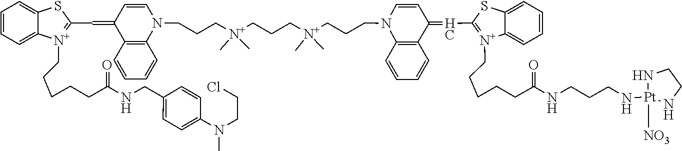 |

In some embodiments, the compound is selected from the compounds of Table 5.

TABLE 5
Selected embodiments of the compounds of the disclosure.

| Cpd # | Structure |
|---|---|
| 1 | 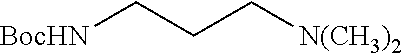 [SMILES: CN(C)CCCNC(=O)OC(C)(C)C] |
| 2 | 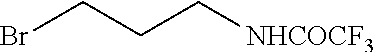 |
| 3 | 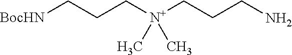 [SMILES: CC(C)(C)OC(=O)NCCC[N+](C)(C)CCCN] |
| 4 | 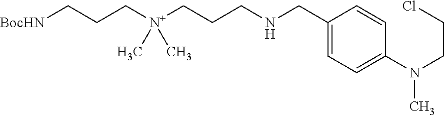 [SMILES: CN(CCCl)c1ccc(CNCCC[N+](C)(C)CCCNC(=O)OC(C)(C)C)cc1] |
| 5 | 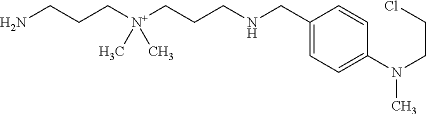 [SMILES: CN(CCCl)c1ccc(CNCCC[N+](C)(C)CCCN)cc1] |
| 6 | 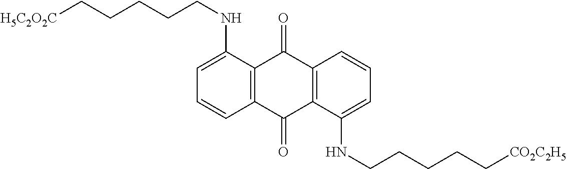 |
| 7 | 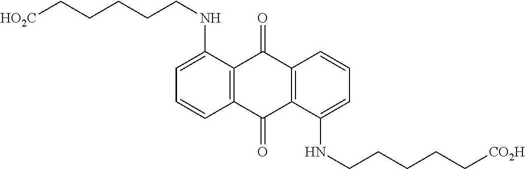 [SMILES: O=C(O)CCCCCNc1cccc2c1C(=O)c1cccc(NCCCCCC(=O)O)c1C2=O] |
| 9 | 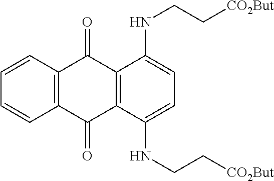 |
| 10 | 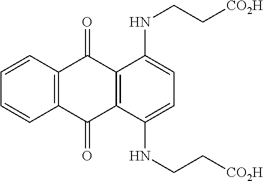 [SMILES: O=C(O)CCNc1ccc(NCCC(=O)O)c2c1C(=O)c1ccccc1C2=O] |
| 12 | 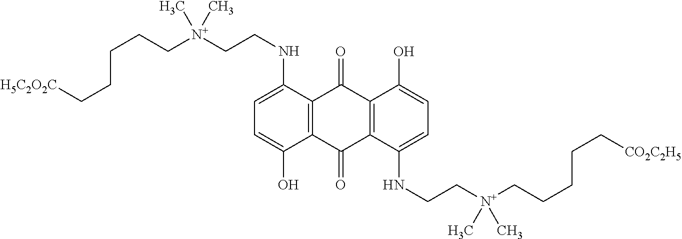 |
| 13 | 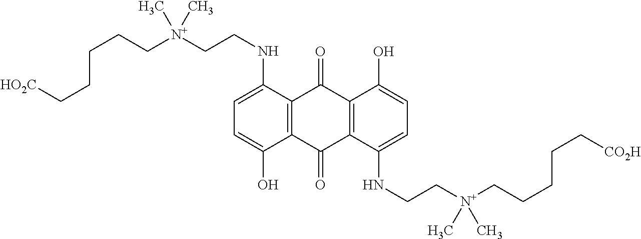 [SMILES: C[N+](C)(CCCCCC(=O)O)CCNc1ccc(O)c2c1C(=O)c1c(O)ccc(NCC[N+](C)(C)CCCCCC(=O)O)c1C2=O] |
| 16 | 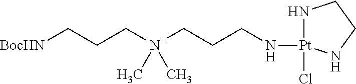 |
| 17 | 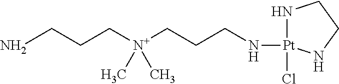 |

In some embodiments, the compounds of the disclosure can be used for detection of nucleic acids in bacteria, viruses, or samples immobilized relative to a matrix or a surface.

In some embodiments, a method of detecting nucleic acids in a sample is disclosed, the method comprising:
a) contacting a sample that contains a nucleic acid with a compound of the present disclosure;
b) incubating the contacted sample under conditions sufficient for the compound to associate with the nucleic acid in the sample to produce a nucleic acid complex that prevents the nucleic acid from being amplified;
c) extracting the nucleic acid;
d) purifying the nucleic acid; and
e) detecting a signal of the amplified nucleic acid.

In some embodiments, the compounds are of Formula 1. In some embodiments, the compounds are of Formula 2. In some embodiments, the compounds are of Formula 3. In some embodiments, the compounds are of Formula 4.

In some embodiments, the nucleic acid is DNA.

In some embodiments, the compounds of the disclosure may be included in a kit. A kit may comprise a compound of the present disclosure, information or a protocol regarding use of the compound or the kit, and/or other useful or necessary materials or reagents, such as any materials or reagents suitable for the detection of nucleic acids, for example, such as a buffer, a detergent, a DNA or RNA standard, a DNA or RNA ladder, and/or matrix.

In some embodiments, a kit for detecting nucleic acids in a sample is disclosed, the kit comprising:
a) a compound of the present disclosure; and
b) one or more components selected from a buffer, a nucleic acid standard, a DNA or RNA ladder, a detergent, a matrix, and an instruction sheet concerning use of the kit for detecting nucleic acids in a sample.

In some embodiments, the compounds are of Formula 1. In some embodiments, the compounds are of Formula 2. In some embodiments, the compounds are of Formula 3. In some embodiments, the compounds are of Formula 4.

In some embodiments, the nucleic acid is DNA.

### EXAMPLES

Examples of some synthetic strategies for selected compounds of the disclosure, as well as their characterization, synthetic precursors, conjugates and methods of use are provided in the examples below. Further modifications and permutations will be obvious to one skilled in the art. The examples below are given so as to illustrate the practice of this disclosure. They are not intended to limit or define the entire scope of this disclosure. It is to be understood that this disclosure is not limited to particular aspects described, as such may vary. It is also to be understood that the terminology used herein is for the purpose of describing particular aspects only and is not intended to be limiting since the scope of the present disclosure will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, representative illustrative methods and materials are now described.

Synthesis of the reactive compounds of the disclosure depends on initial preparation of certain key intermediates as illustrated in the following examples. For simplicity, all but a few of the possible substituents are shown as hydrogen. These basic structures are optionally further substituted, during or after synthesis, to give the corresponding compound conjugate substituents as defined above. It is recognized that there are many possible variations that may yield equivalent results.

The following abbreviations have the definitions set forth below:

| | |
|---|---|
| PCR | Polymerase chain reaction |
| PMA | Propidium monoazide |
| VPCR | Viability PCR |
| IVD | In vitro diagnostics |
| THF | Tetrahydrafuran |
| DMF | N,N-Dimethylformamide |
| HPLC | High-performance liquid chromatography |
| TSTU | O-(N-Succinimidyl)-N,N,N′,N′-tetramethyluronium |
| | tetrafluoroborate |
| TFA | Trifluoroacetic acid |
| DIEA | N,N-Diisopropylethylamine |
| DMSO | Dimethyl sulfoxide |
| NHS | N-Hydroxysuccinimide |

### Example 1. Preparation of Compound 1

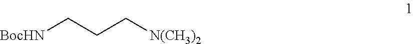 [SMILES: CN(C)CCCNC(=O)OC(C)(C)C]

Di-t-butyl decarbonate (9 g) in anhydrous THF (33 mL) is cooled to 0° C., and 3-(dimethylamino)-1-propylamine (3.2 g) is added slowly, followed by the addition of triethylamine (4.5 mL). The reaction mixture is stirred at room temperature for 16 hours. THF is evaporated and the residue is diluted with diethyl ether. The solution is washed with 1M NaOH and brine, dried over anhydrous Na₂SO₄. The solution is concentrated to give Compound 1.

### Example 2. Preparation of Compound 2

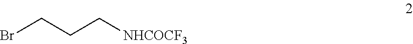

3-Bromopropylamine hydrobromide (7.6 g) in methanol (50 mL) is cooled to 0° C., and methyl trifluoroacetate (4.0 mL) is added followed by triethylamine (5.0 mL). The reaction mixture is stirred at room temperature for 3 hours. Methanol is evaporated and the residue is diluted with diethyl ether. The solution is washed with 0.2M KH₂PO₄ and brine, dried over anhydrous Na₂SO₄. The solution is concentrated to give white solid Compound 2.

### Example 3. Preparation of Compound 3

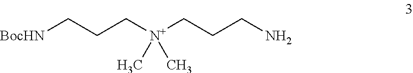 [SMILES: CC(C)(C)OC(=O)NCCC[N+](C)(C)CCCN]

Compounds 1 (6.9 g) and 2 (8.1 g) are dissolved in DMF (5 mL), and the solution is stirred at 50° C. for 48 hours. The reaction mixture is added into diethyl ether (100 mL) and centrifuged. The sticky oil is washed with diethyl ether twice and dried under vacuum. To the sticky oil are added methanol (50 mL) and 28% ammonium hydroxide (50 mL). The reaction mixture is stirred at 30° C. for 30 hours. Methanol is evaporated and the residue is lyophilized to give a sticky oil. The oil is further washed with diethyl ether twice and dried under vacuum to afford Compound 3 as a sticky white solid.

### Example 4. Preparation of Compound 4

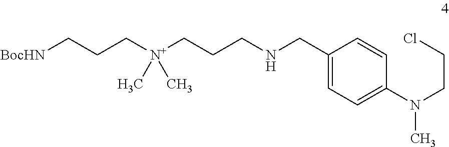 [SMILES: CN(CCCl)c1ccc(CNCCC[N+](C)(C)CCCNC(=O)OC(C)(C)C)cc1]

Compounds 3 (6.3 g) and 4-((2-chloroethyl)(methyl)amino)benzaldehyde (1.3 g) are dissolved in methanol (50 mL), and to the methanol solution of Compound 3 p-toluenesulfonic acid (42 mg) is added. The reaction mixture is stirred at room temperature for 24 hours. NaBH₃CN (440 mg; 7.00 mmol) is added and stirred at room temperature for 4 hours. Methanol is evaporated and the residue is purified by a flash column chromatography of silica gel using methanol-CH₂Cl₂ (1:7) as the eluent to afford Compound 4 as a yellow solid.

### Example 5. Preparation of Compound 5

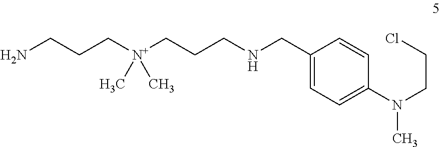 [SMILES: CN(CCCl)c1ccc(CNCCC[N+](C)(C)CCCN)cc1]

To 4M HCl in dioxane (15 mL) Compound 4 (1.18 g) is added, and the reaction mixture is stirred at room temperature for 1 hour. The liquid layer is discarded and the solid is dissolved in water (20 mL). The crude mixture is purified by a reverse-phase Prep HPLC to afford Compound 5.

### Example 6. Preparation of Compound 6

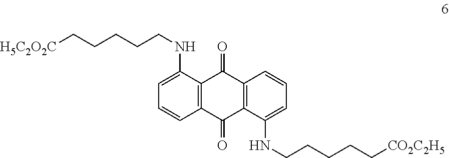

To a stirred solution of 1,5-diaminoanthraquinone (1.13 g) in DMF (10 mL) ethyl 6-bromohexanoate (9.6 mL) and K₂CO₃ (3.5 g) are added. The reaction mixture is stirred at 140° C. for 18 hours. Ethyl acetate (100 mL) is added, and the resulting solution is washed with water and brine, dried over anhydrous Na₂SO₄. The solution is concentrated, and the residue is purified by a flash column chromatography of silica gel using ethyl acetate-hexane (1:3) as the eluent to afford Compound 6 as dark red solid.

### Example 7. Preparation of Compound 7

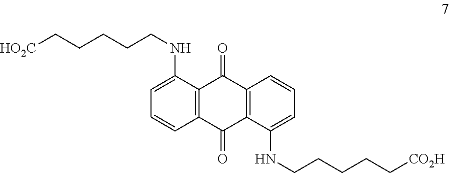 [SMILES: O=C(O)CCCCCNc1cccc2c1C(=O)c1cccc(NCCCCCC(=O)O)c1C2=O]

Compound 6 (1.41 g) and 1M NaOH (aq) (30 mL) are dissolved in methanol (20 mL). The reaction mixture is stirred at 40° C. for 50 hours and neutralized with 4 M HCl until pH=7.0. The crude solution is purified by a reverse-phase Prep HPLC to afford Compound 7 as dark red powder.

### Example 8. Preparation of Compound 8

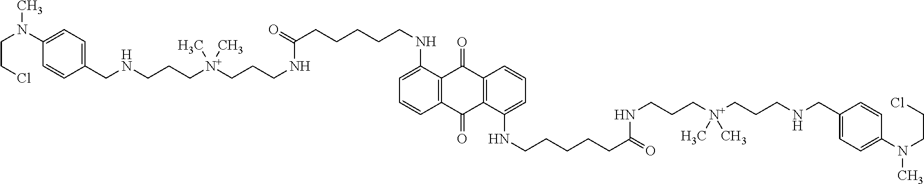 [SMILES: CN(CCCl)c1ccc(CNCCC[N+](C)(C)CCCNC(=O)CCCCCNc2cccc3c2C(=O)c2cccc(NCCCCCC(=O)NCCC[N+](C)(C)CCCNCc4ccc(N(C)CCCl)cc4)c2C3=O)cc1]

Compound 7 (14 mg) and Et₃N (5 μL; 36 μmol) are dissolved in anhydrous DMF (200 μL). TSTU (14 mg) is added, and the resulted reaction mixture is stirred at room temperature for 1 hour. The reaction mixture is added dropwise into the solution of Compound 5 (44 mg) and DIEA (50 μL) in anhydrous DMF (0.8 mL). The reaction mixture is stirred at room temperature for 2 hours. To the reaction mixture is added 1:4 TFA-water (1.0 mL), and the resulted reaction mixture is purified by a reverse-phase Prep HPLC to afford Compound 8 as dark red solid.

### Example 9. Preparation of Compound 9

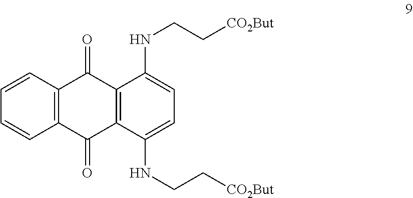

1,4-Difluoroanthraquinone (442 mg), β-alanine t-butyl ester hydrochloride (1.34 g) and DIEA (2.1 mL) are dissolved in anhydrous DMSO (5.5 mL). The reaction mixture is stirred at 110° C. for 9 hours and diluted with ethyl acetate (100 mL). The solution is washed with water and brine, dried over anhydrous Na₂SO₄. The resulted solution is concentrated, and the residue is purified by a flash column chromatography of silica gel using ethyl acetate-hexane (1:3) as the eluent to afford Compound 9 as blue solid.

### Example 10. Preparation of Compound 10

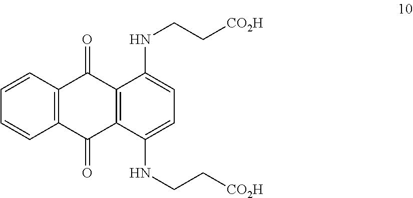 [SMILES: O=C(O)CCNc1ccc(NCCC(=O)O)c2c1C(=O)c1ccccc1C2=O]

Compound 9 (675 mg; 1.36 mmol) and trifluoroacetic acid (3 mL) are added into CH₂CL₂ (3 mL). The reaction mixture is stirred at room temperature for 1.5 hours. The reaction mixture is added into diethyl ether (60 mL) and centrifuged. The solid is washed with ether and dried under vacuum to afford Compound 10 as blue solid.

### Example 11. Preparation of Compound 11

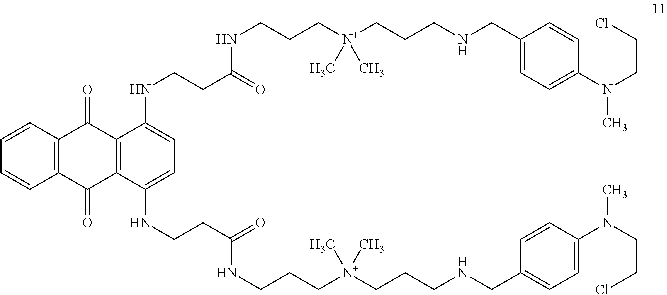 [SMILES: CN(CCCl)c1ccc(CNCCC[N+](C)(C)CCCNC(=O)CCNc2ccc(NCCC(=O)NCCC[N+](C)(C)CCCNCc3ccc(N(C)CCCl)cc3)c3c2C(=O)c2ccccc2C3=O)cc1]

Compound 11 is synthesized from the reaction of Compound 10 with Compound 5 analogously to the procedure of Compound 8.

### Example 12. Preparation of Compound 12

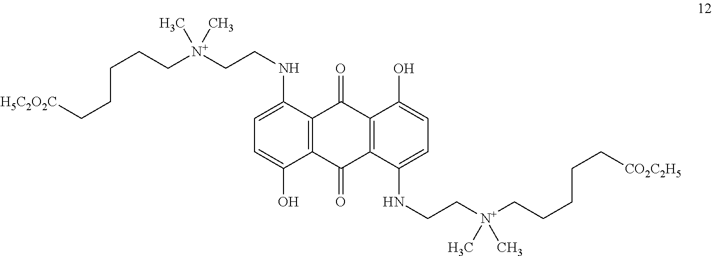

Compound 12 is analogously synthesized from the reaction of 1,5-bis((2-(dimethylamino)ethyl)amino)-4,8-dihydroxyanthracene-9,10-dione with ethyl 6-bromohexanoate according to the procedure of Compound 3.

### Example 13. Preparation of Compound 13

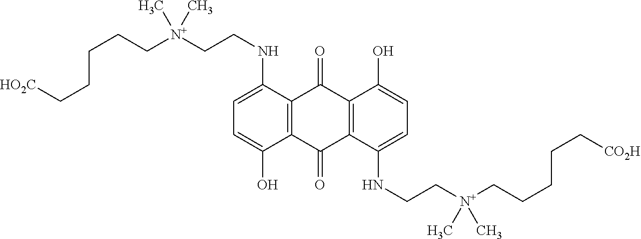 [SMILES: C[N+](C)(CCCCCC(=O)O)CCNc1ccc(O)c2c1C(=O)c1c(O)ccc(NCC[N+](C)(C)CCCCCC(=O)O)c1C2=O]

Compound 13 is synthesized from Compound 12 analogously according to the procedure of Compound 7.

### Example 14. Preparation of Compound 14

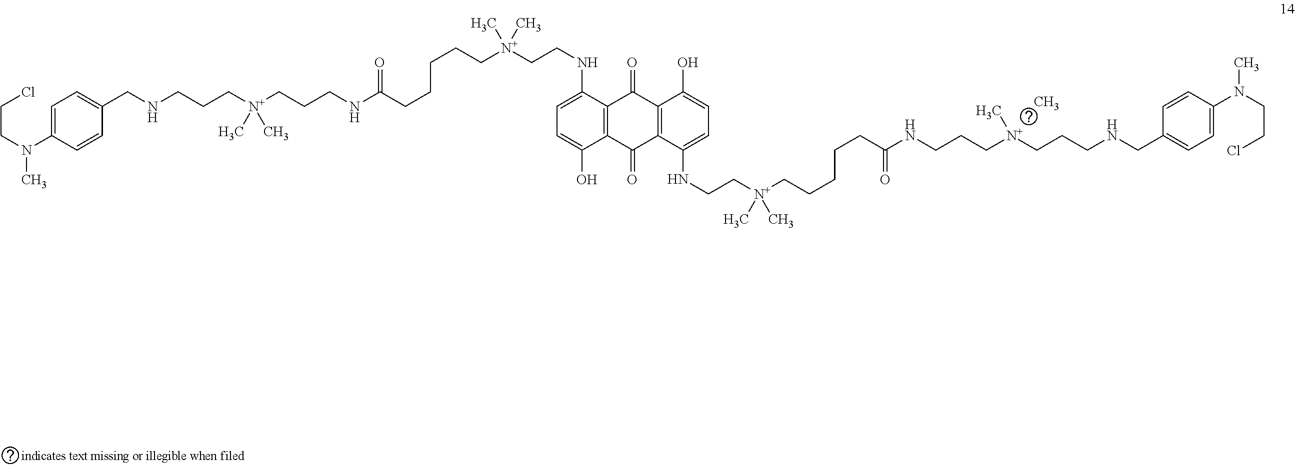

Compound 14 is synthesized from the reaction of Compound 13 with Compound 5 analogously according to the procedure of Compound 8.

### Example 15. Preparation of Compound 15

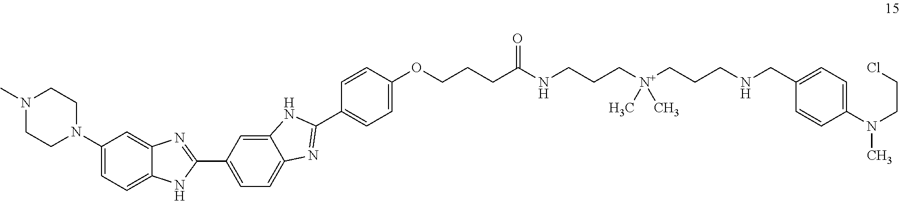 [SMILES: CN1CCN(c2ccc3[nH]c(-c4ccc5nc(-c6ccc(OCCCC(=O)NCCC[N+](C)(C)CCCNCc7ccc(N(C)CCCl)cc7)cc6)[nH]c5c4)nc3c2)CC1]

To the solution of Compound 5 (44 mg) and DIEA (50 μL) in anhydrous DMF (0.8 mL) is dropwise added Hoechst 33342 NHS ester (50 mg). The reaction mixture is stirred at room temperature for 2 hours. To the reaction mixture is added 1:4 TFA-water (1.0 mL), and the resulted reaction mixture is purified by a reverse-phase Prep HPLC to afford Compound 15.

### Example 16. Preparation of Compound 16

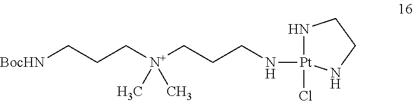

Compound 16 is analogously prepared from the reaction of Compound 3 with platinum(ethylenediamine) chloride according to the procedure described U.S. Pat. No. 6,133,038.

### Example 17. Preparation of Compound 17

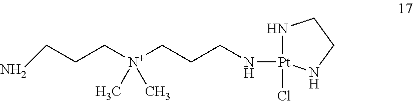

To 4M HCl in dioxane (15 mL) Compound 16 (100 mg) is added, and the reaction mixture is stirred at room temperature for 1 hour. The liquid layer is discarded and the solid is dissolved in water (20 mL). The crude mixture is purified by a reverse-phase Prep HPLC to afford Compound 17.

### Example 18. Preparation of Compound 18

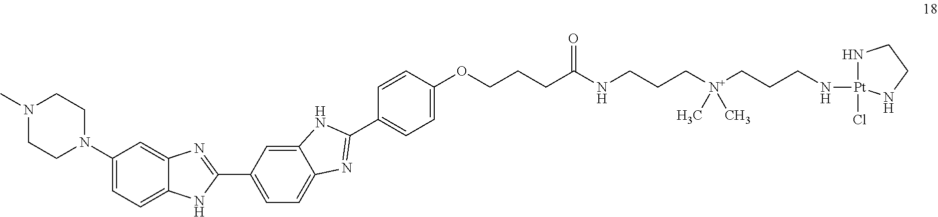

Compound 18 is synthesized from the reaction of Hoechst 33342 NHS ester with Compound 17 analogously according to the procedure of Compound 15.

The additional compounds of the disclosure can be analogously prepared as illustrated in the above examples. The additional examples are listed in the following table.

TABLE 6
Selected compounds of the disclosure.

| Cpd | |
|---|---|
| # | Structure |
| 20 | 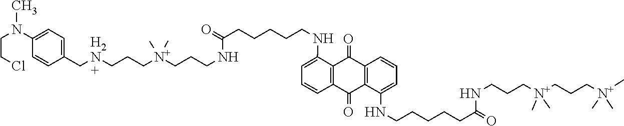 |
| 21 | 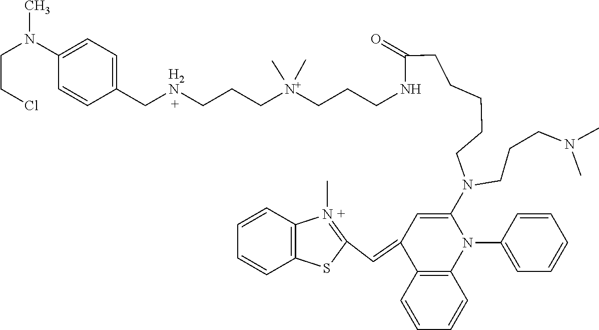 |
| 22 | 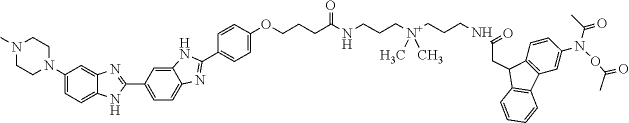 [SMILES: CC(=O)ON(C(C)=O)c1ccc2c(c1)-c1ccccc1C2CC(=O)NCCC[N+](C)(C)CCCNC(=O)CCCOc1ccc(-c2nc3ccc(-c4nc5cc(N6CCN(C)CC6)ccc5[nH]4)cc3[nH]2)cc1] |
| 23 | 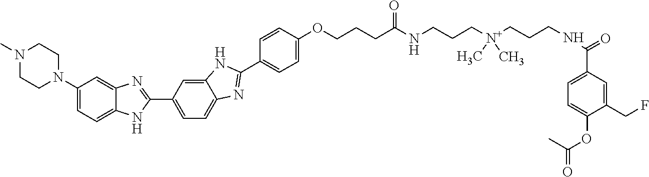 [SMILES: CC(=O)Oc1ccc(C(=O)NCCC[N+](C)(C)CCCNC(=O)CCCOc2ccc(-c3nc4ccc(-c5nc6cc(N7CCN(C)CC7)ccc6[nH]5)cc4[nH]3)cc2)cc1CF] |
| 24 | 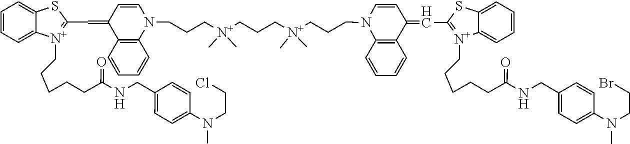 [SMILES: CN(CCCl)c1ccc(CNC(=O)CCCCC[n+]2c(C=C3C=CN(CCC[N+](C)(C)CCC[N+](C)(C)CCCN4C=CC(=Cc5sc6ccccc6[n+]5CCCCCC(=O)NCc5ccc(N(C)CCBr)cc5)c5ccccc54)c4ccccc43)sc3ccccc32)cc1] |
| 25 | 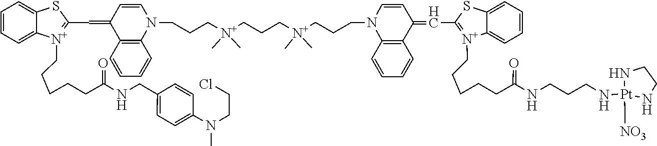 |
| 26 | 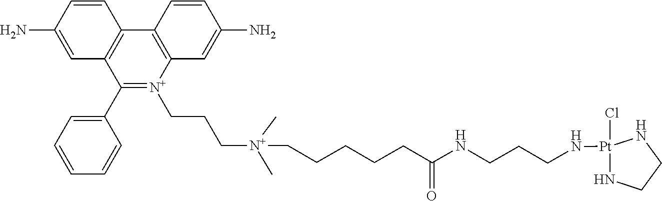 |
| 27 | 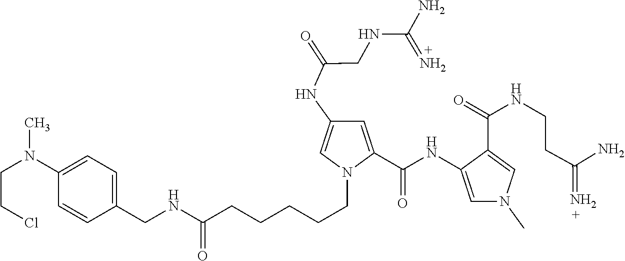 [SMILES: CN(CCCl)c1ccc(CNC(=O)CCCCCn2cc(NC(=O)CNC(=N)N)cc2C(=O)Nc2cn(C)cc2C(=O)NCCC(=N)N)cc1] |
| 28 | 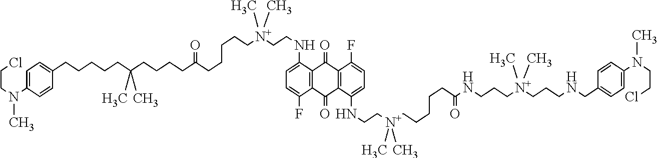 [SMILES: CN(CCCl)c1ccc(CCCCCC(C)(C)CCCCC(=O)CCCCC[N+](C)(C)CCNc2ccc(F)c3c2C(=O)c2c(F)ccc(NCC[N+](C)(C)CCCCCC(=O)NCCC[N+](C)(C)CCCNCc4ccc(N(C)CCCl)cc4)c2C3=O)cc1] |
| 29 | 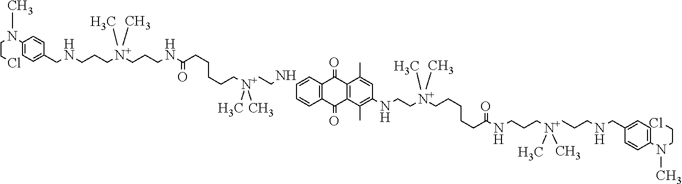 [SMILES: Cc1cc(NCC[N+](C)(C)CCCCCC(=O)NCCC[N+](C)(C)CCCNCc2ccc(N(C)CCCl)cc2)c(C)c2c1C(=O)c1cc(NCC[N+](C)(C)CCCCCC(=O)NCCC[N+](C)(C)CCCNCc3ccc(N(C)CCCl)cc3)ccc1C2=O] |
| 30 | 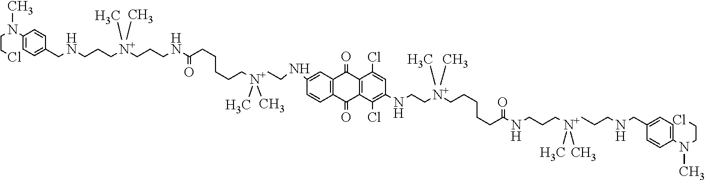 [SMILES: CN(CCCl)c1ccc(CNCCC[N+](C)(C)CCCNC(=O)CCCCC[N+](C)(C)CCNc2ccc3c(c2)C(=O)c2c(Cl)cc(NCC[N+](C)(C)CCCCCC(=O)NCCC[N+](C)(C)CCCNCc4ccc(N(C)CCCl)cc4)c(Cl)c2C3=O)cc1] |
| 31 | 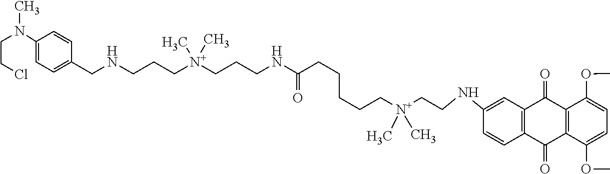 [SMILES: COc1ccc(OC)c2c1C(=O)c1ccc(NCC[N+](C)(C)CCCCCC(=O)NCCC[N+](C)(C)CCCNCc3ccc(N(C)CCCl)cc3)cc1C2=O] |
| 32 | 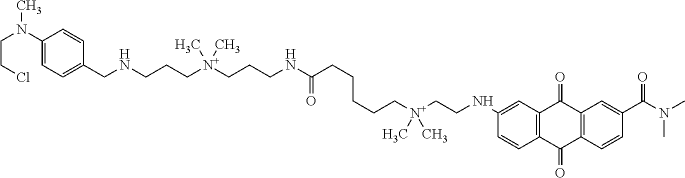 [SMILES: CN(C)C(=O)c1ccc2c(c1)C(=O)c1cc(NCC[N+](C)(C)CCCCCC(=O)NCCC[N+](C)(C)CCCNCc3ccc(N(C)CCCl)cc3)ccc1C2=O] |
| 33 | 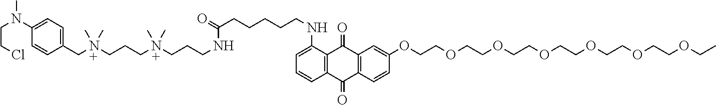 |

### Example 19. The Use of the Compounds for Viability PCR

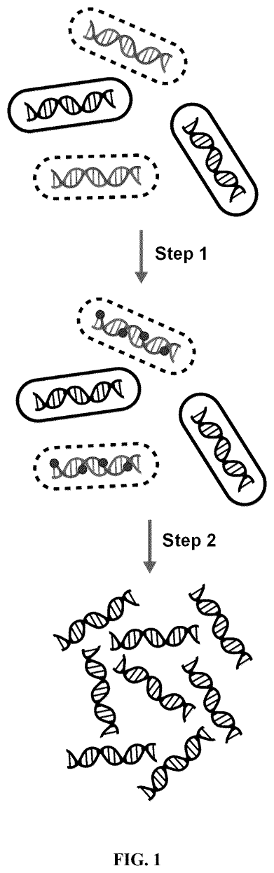

E. coli bacteria (ATCC 25922) are grown overnight. Next day, 600 μL of cell culture is used and aliquoted into a set of tubes (100 μL/tube). The dead cells are produced by heating live E. coli cells for 10 minutes at 90° C. PMA or a selected compound of the disclosure (e.g., Compound 8) are respectively added to the live and dead cells at the final concentration of 50 μM. They are incubated in the dark for 20 minutes at room temperature. The cells are centrifuged (live at 3000 rpm for 5 minutes and dead cells at 10000 rpm for 5 minutes). The supernatants are removed, and cells are used to extract genomic DNA using Qiagen kit. DNA is eluted with 200 μL of elution buffer. The samples are used for viability PCR tests. Final concentration of Forward primer (5′-TGGATCGCGAAAACTGTGGA-3′, 400 nM) (SEQ ID NO:1) and Reverse primer (5′-CGGTGATATCGTCCACCCAG-3′, 400 nM) (SEQ ID NO:2) are used in the qPCR master mix. 10 μL of genomic DNA (eluted sample from 200 μL) and 10 μL of 2× master mix containing both primers are added into a PCR plate and PCR is performed as per Thermal Cycling parameters as shown below. The results are summarized in FIG. 1.

TABLE 7
Thermal cycling parameters.

| | |
|---|---|
| Polymerase | |
| Activation | PCR (30-40 cycles) |

| Parameter | Hold | Denature | Anneal/Extend |
|---|---|---|---|
| Temperature | 95 °C | 95 °C | 60 °C |
| Time (minute) | 30 | 20 | 30 |

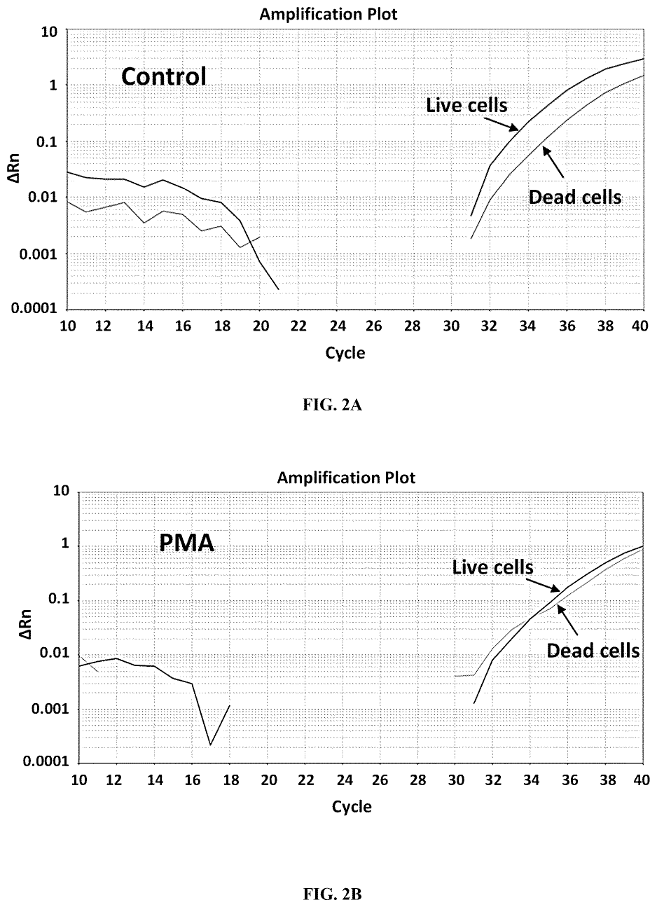

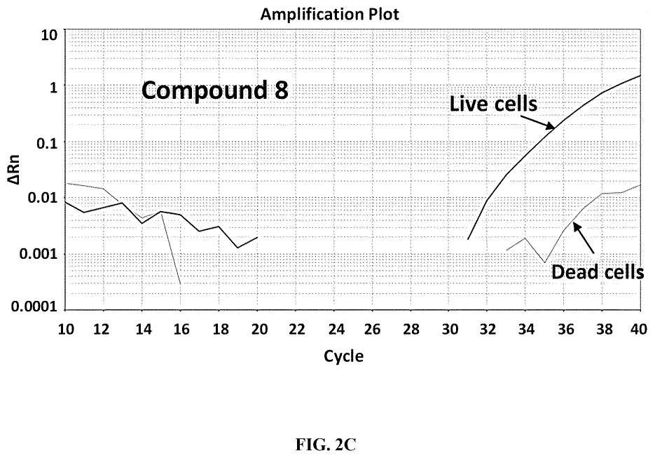

FIGS. 2A-2C present normalized qPCR curves from a viability PCR experiment in which live, and heat-inactivated E. coli are treated without an inhibitor (control), PMA (with UV light irradiation) or Compound 8 (without light activation). qPCR is performed using primers against a region of the uidA gene. The treatment of Compound 8 has no effect on amplification of DNA from live E. coli but causes a dramatic delay in amplification of DNA from heat-killed E. coli. On the contrary, in the absence of light, PMA does not have any effect on the amplification of DNA from heat-killed E. coli.

As shown in the above examples, useful methods of preparing any of various compounds described herein and useful methods of using any of these compounds have also been described. Useful kits suitable for determining nucleic acids, which comprises a suitable compound described herein, have also been described. Various modifications, processes, as well as numerous structures relating to the description herein may be applicable, as they are obvious to those of ordinary skill in the art, upon review of the specification. Various aspects and features may have been explained or described in relation to understandings, beliefs, theories, underlying assumptions, and/or working or prophetic examples, although it will be understood that any such understanding, belief, theory, underlying assumption, and/or working or prophetic example is not binding. Although the various aspects and features have been described with respect to various embodiments and examples herein, it will be understood that any of the same is not limiting with respect to the full scope of the claims.

1. A compound of Formula 1:
[BRM1-L1]ₘ-NBM-[L2-BRM2]ₙ Formula 1
or a salt thereof, wherein:
BRM1 and BRM2 are independently a nucleic acid base-reactive moiety;
NBM is a nucleic acid binding moiety;
L1 and L2 are independently a linker; and
m and n are independently an integer from 0 to 4, wherein the sum of m and n is from 1 to 4.

2. The compound of claim 1, wherein the sum of m and n is 1.

3. The compound of claim 1, wherein the sum of m and n is 2.

4. The compound of claim 3, wherein m and n are each 1.

5. The compound of claim 1, wherein m and n are independently 1 or 2.

6. The compound of claim 1, wherein BRM1 and BRM2 are independently selected from a 2-chloroethylamino moiety, a 2-bromoethylamino moiety, a 2-iodoethylamino moiety, an aziridinyl, an enediynyl, a psoralen, an azidoaryl, a quinomethide-generating moiety, a N-acetoxy-N-2-acetylaminofluorenyl, a 4-acetoxy-3-fluoromethyl-phenyl moiety, and a platinum complex.

7. (canceled)

8. The compound of claim 6, BRM1 and BRM2 are each independently selected from Cl—C(R₁R₂)C(R₃R₄)N(R₅)—, Br—C(R₁R₂)C(R₃R₄)N(R₅)—, and I—C(R₁R₂)C(R₃R₄)N(R₅)—, wherein R₁ to R₅ are independently selected from H, optionally substituted alkyl, aryl, and heteroaryl.

9. The compound of claim 6, wherein BRM1 and BRM2 are each independently selected from:

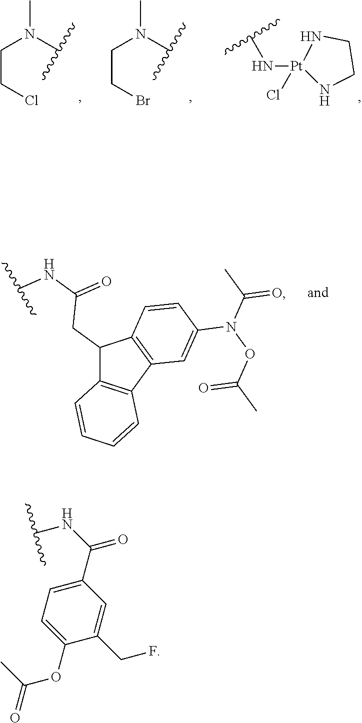

10. The compound of claim 1, wherein NBM is an intercalator or a minor groove binding moiety.

11. The compound of claim 1, wherein NBM is selected from a phenanthridinum moiety, a proflavine, an anthraquinoid moiety, an actinomycin D moiety, an acridine, a netropsin moiety, a distamycin moiety, a Hoechst moiety, a beranil moiety, and a DAPI moiety.

12-15. (canceled)

16. The compound of claim 1, wherein L1 and L2 are independently a linear linker having a backbone of 4 or more consecutive atoms, comprising one or more divalent linking moieties independently selected from —C₁₋₂₀-alkylene-, —NHCO—, —CONH—, —NH—, —⁺NH₂—, —NMe-, —⁺NMe₂-, —NHCONH—, —NHCSNH—, —O(CH₂)ₚ—, —(OCH₂CH₂)ₚ—, —NHSO₂—, —SO₂NH—, —CO—, —SO₂—, —O—, and —S—, wherein each p is independently 1 to 10.

17-18. (canceled)

19. The compound of claim 16, wherein L1 and L2 are independently selected from:

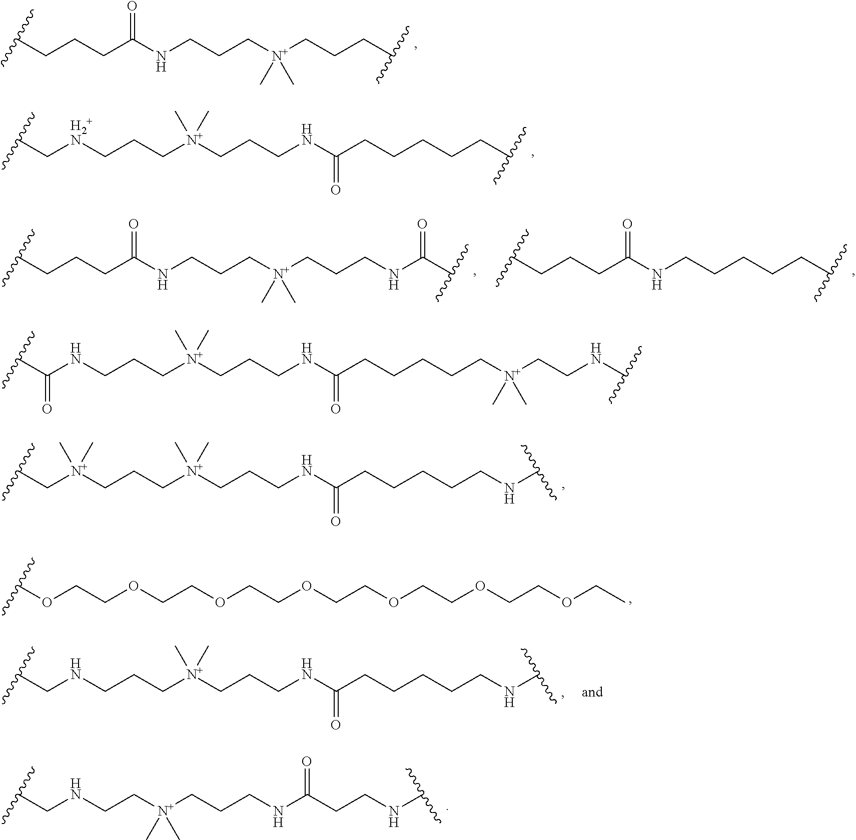

20. The compound of claim 1, wherein:
L1 and L2 independently comprise a polyethylene glycol (PEG) linking moiety;
L1 and L2 independently comprise a polyamine linking moiety —[N⁺(R)₂—C₍₁₋₆₎alkyl]ₙ- wherein R is H or methyl and n is 1-6, or a polyammonium linking moiety —[NR—C₁₋₆alkyl]ₙ- wherein R is H or methyl and n is 1-6;
L1 and L2 independently comprise an oxo and/or arylene; and/or
L1 and L2 are independently represented by Formula 1′: 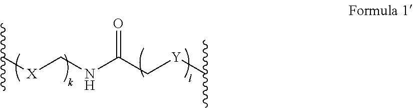
wherein:
each X and each Y are independently selected from optionally substituted alkyl, ether, amino group, amide group, oxo, ammonium, aryl, and heteroaryl; and
k and l are independently an integer from 0 to 10.

21-26. (canceled)

27. The compound of claim 4, wherein:
NBM is an anthraquinoid moiety;
m and n are each 1; and
BRM1 and BRM2 are each a 2-chloroethylamino moiety.

28. A compound having Formula 2: 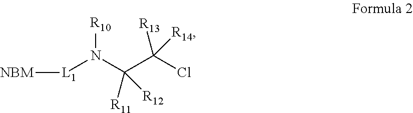
or a salt thereof, wherein:
NBM is selected from an intercalator and a minor groove binding moiety; and
R₁₀-R₁₄ are independently selected from H, an alkyl, an alkoxy, an aryloxy, an aryl, and a heteroaryl; and
L1 is a linker comprising 1-100 non-hydrogen atoms selected from C, N, O, P, and S, further comprising a plurality of single, double, triple or aromatic carbon-carbon bonds, carbon-nitrogen bonds, nitrogen-nitrogen bonds, carbon-oxygen bonds, carbon-sulfur bonds, phosphorus-oxygen bonds, phosphorus-nitrogen bonds, and combination thereof.

29. The compound of claim 28, wherein:
NBM is selected from a phenanthridinum moiety, a proflavine, an anthraquinoid moiety, an actinomycin D moiety, an acridine, a netropsin moiety, a distamycin moiety, a Hoechst moiety, a beranil moiety, and a DAPI moiety; and
L1 is represented by Formula 1′: 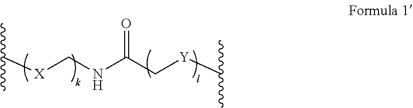
wherein:
each X and each Y are independently selected from optionally substituted alkyl, amino group, and ammonium, and
k and l are independently an integer from 1 to 6.

30-31. (canceled)
32. (Original) The compound of claim 28, wherein the compound is selected from:

| Cpd # | Structure |
|---|---|
| 15 | 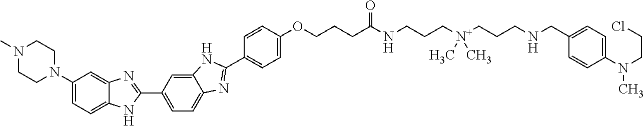 [SMILES: CN1CCN(c2ccc3[nH]c(-c4ccc5nc(-c6ccc(OCCCC(=O)NCCC[N+](C)(C)CCCNCc7ccc(N(C)CCCl)cc7)cc6)[nH]c5c4)nc3c2)CC1] |
| 18 | 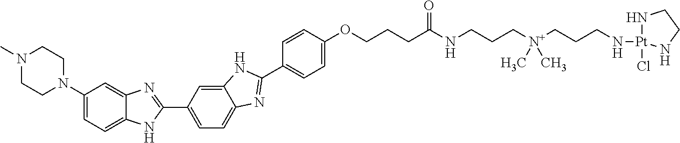 |
| 20 | 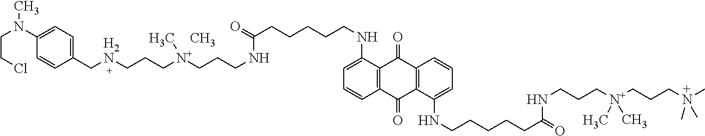 |
| 21 | 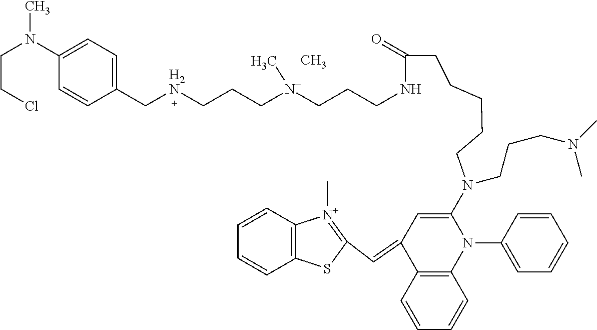 |
| 22 | 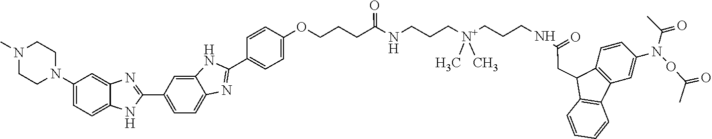 [SMILES: CC(=O)ON(C(C)=O)c1ccc2c(c1)-c1ccccc1C2CC(=O)NCCC[N+](C)(C)CCCNC(=O)CCCOc1ccc(-c2nc3ccc(-c4nc5cc(N6CCN(C)CC6)ccc5[nH]4)cc3[nH]2)cc1] |
| 23 | 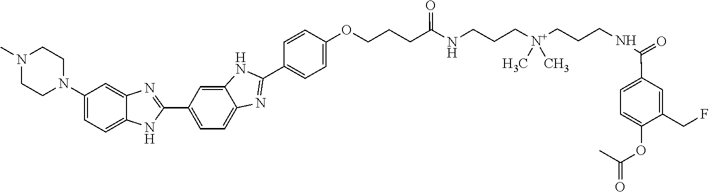 [SMILES: CC(=O)Oc1ccc(C(=O)NCCC[N+](C)(C)CCCNC(=O)CCCOc2ccc(-c3nc4ccc(-c5nc6cc(N7CCN(C)CC7)ccc6[nH]5)cc4[nH]3)cc2)cc1CF] |
| 26 | 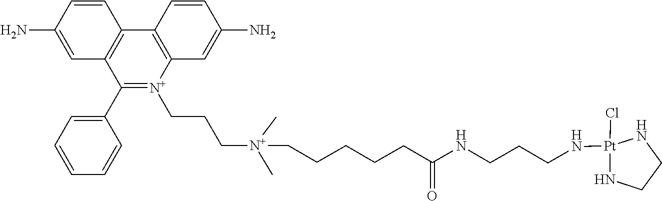 |
| 27 | 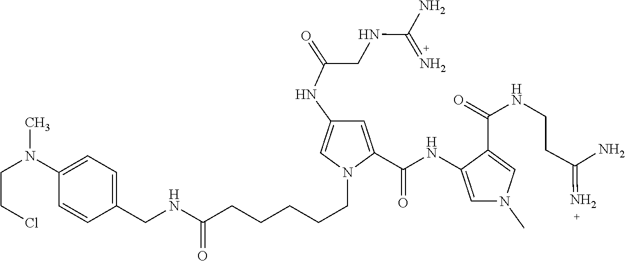 [SMILES: CN(CCCl)c1ccc(CNC(=O)CCCCCn2cc(NC(=O)CNC(=N)N)cc2C(=O)Nc2cn(C)cc2C(=O)NCCC(=N)N)cc1] |
| 31 | 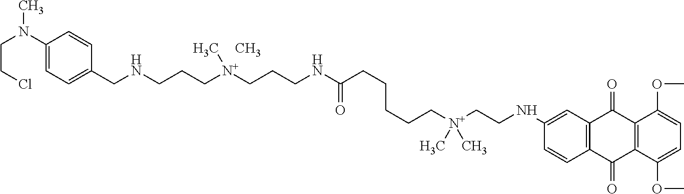 |
| 32 | 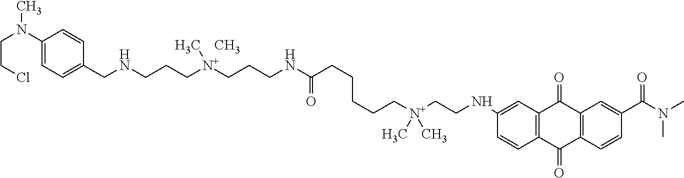 |
| 33 | 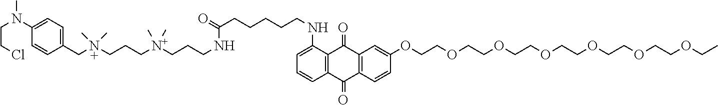 |

33. A compound having Formula 3: 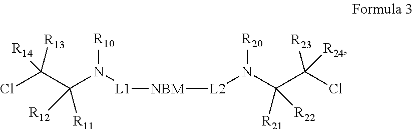
or a salt thereof, wherein:
NBM is selected from an intercalator and a minor groove binding moiety;
R₁₀-R₁₄ and R₂₀-R₂₄ are independently selected from H, an alkyl, an alkoxy, an aryloxy, an aryl, and a heteroaryl, each optionally substituted; and
L1 and L2 are each independently a covalent linkage having 1-100 non-hydrogen atoms selected from C, N, O, P, and S, further comprising a plurality of single, double, triple or aromatic carbon-carbon bonds, carbon-nitrogen bonds, nitrogen-nitrogen bonds, carbon-oxygen bonds, carbon-sulfur bonds, phosphorus-oxygen bonds, phosphorus-nitrogen bonds, and combinations thereof.

34. The compound of claim 33, wherein:
NBM is selected from a phenanthridinum moiety, a proflavine, an anthraquinoid moiety, an actinomycin D moiety, an acridine, a netropsin moiety, a distamycin moiety, a Hoechst moiety, a beranil moiety, and a DAPI moiety; and
L1 and L2 are each independently represented by Formula 1′: 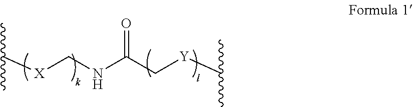
wherein:
each X and each Y are independently selected from optionally substituted alkyl, amino group, and ammonium; and
k and l are independently an integer from 1 to 6.

35-36. (canceled)

37. The compound of claim 33, wherein the compound is selected from:

| Cpd | |
|---|---|
| # | Structure |
| 8 | 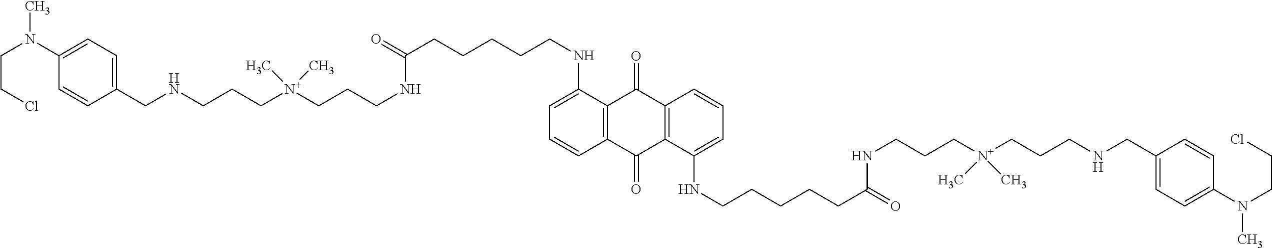 [SMILES: CN(CCCl)c1ccc(CNCCC[N+](C)(C)CCCNC(=O)CCCCCNc2cccc3c2C(=O)c2cccc(NCCCCCC(=O)NCCC[N+](C)(C)CCCNCc4ccc(N(C)CCCl)cc4)c2C3=O)cc1] |
| 11 | 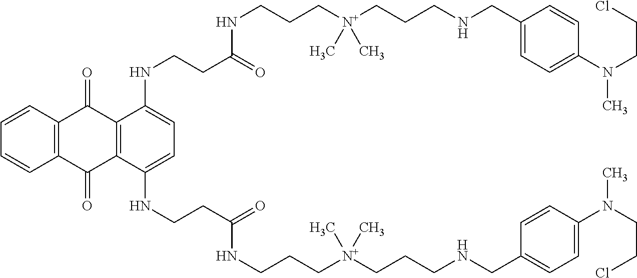 [SMILES: CN(CCCl)c1ccc(CNCCC[N+](C)(C)CCCNC(=O)CCNc2ccc(NCCC(=O)NCCC[N+](C)(C)CCCNCc3ccc(N(C)CCCl)cc3)c3c2C(=O)c2ccccc2C3=O)cc1] |
| 14 | 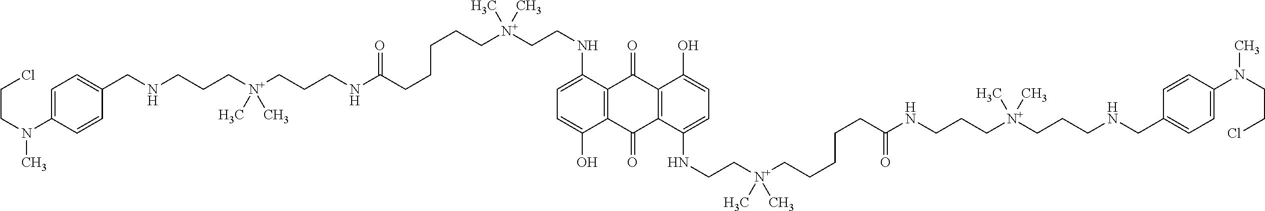 [SMILES: CN(CCCl)c1ccc(CNCCC[N+](C)(C)CCCNC(=O)CCCCC[N+](C)(C)CCNc2ccc(O)c3c2C(=O)c2c(O)ccc(NCC[N+](C)(C)CCCCCC(=O)NCCC[N+](C)(C)CCCNCc4ccc(N(C)CCCl)cc4)c2C3=O)cc1] |
| 28 | 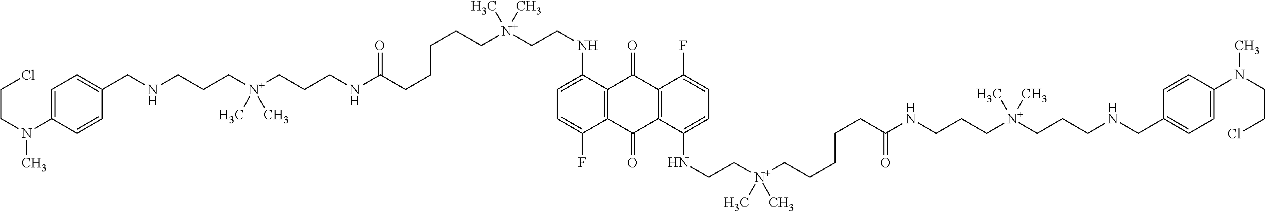 [SMILES: CN(CCCl)c1ccc(CNCCC[N+](C)(C)CCCNC(=O)CCCCC[N+](C)(C)CCNc2ccc(F)c3c2C(=O)c2c(F)ccc(NCC[N+](C)(C)CCCCCC(=O)NCCC[N+](C)(C)CCCNCc4ccc(N(C)CCCl)cc4)c2C3=O)cc1] |
| 29 | 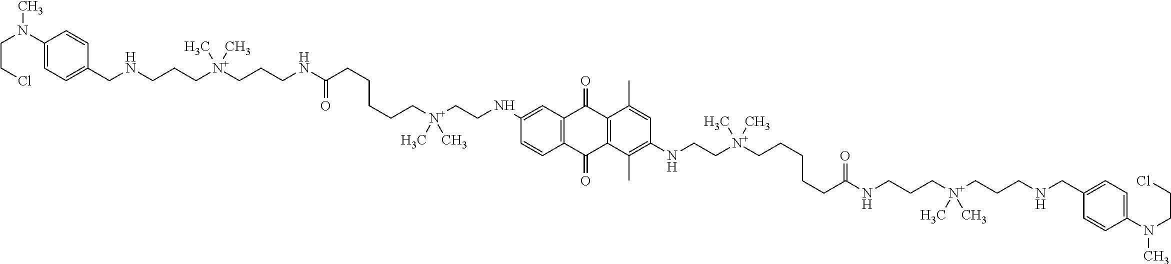 [SMILES: Cc1cc(NCC[N+](C)(C)CCCCCC(=O)NCCC[N+](C)(C)CCCNCc2ccc(N(C)CCCl)cc2)c(C)c2c1C(=O)c1cc(NCC[N+](C)(C)CCCCCC(=O)NCCC[N+](C)(C)CCCNCc3ccc(N(C)CCCl)cc3)ccc1C2=O] |
| 30 | 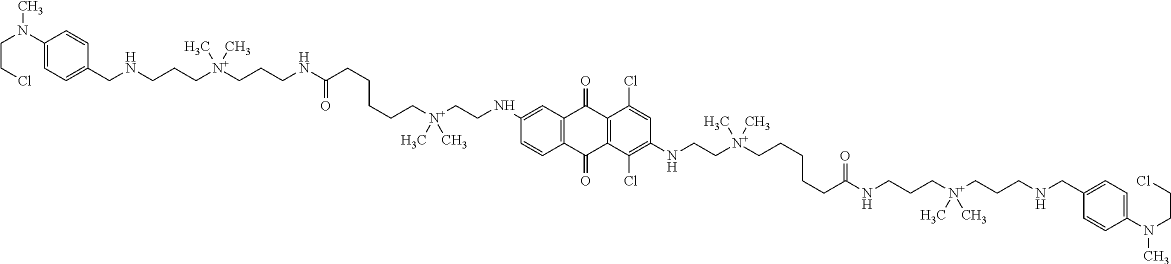 [SMILES: CN(CCCl)c1ccc(CNCCC[N+](C)(C)CCCNC(=O)CCCCC[N+](C)(C)CCNc2ccc3c(c2)C(=O)c2c(Cl)cc(NCC[N+](C)(C)CCCCCC(=O)NCCC[N+](C)(C)CCCNCc4ccc(N(C)CCCl)cc4)c(Cl)c2C3=O)cc1] |

38. A compound having Formula 4: 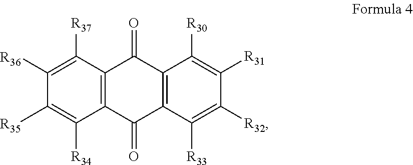
or a salt thereof, wherein:
R₃₀-R₃₇ are independently selected from H, halogen, hydroxy, an optionally substituted amino, optionally substituted alkyl, optionally substituted alkoxy, optionally substituted aryloxy, optionally substituted aryl, optionally substituted heteroaryl and L-BRM, wherein at least one of R₃₀-R₃₇ is a L-BRM, wherein BRM is a nucleic acid base-reactive moiety and L is a linker.

39. The compound of claim 38, wherein BRM is selected from a 2-chloroethylamino moiety, 2-bromoethylamino moiety, 2-iodoethylamino moiety, an aziridinyl, an enediynyl, a psoralen, an azidoaryl, a quinomethide-generating moiety, a N-acetoxy-N-2-acetylaminofluorenyl, and a platinum complex.

40. The compound of claim 38, wherein BRM is selected from Cl—C(R₁R₂)C(R₃R₄)N(R₅)—, Br—C(R₁R₂)C(R₃R₄)N(R₅)—, and I—C(R₁R₂)C(R₃R₄)N(R₅)—, wherein R₁ to R₅ are each independently selected from H, optionally substituted alkyl, optionally substituted aryl, and optionally substituted heteroaryl.

41. The compound of claim 38, wherein:
a) R₃₀ and R₃₃ are independently L-BRM; and
R₃₁, R₃₂ and R₃₄-R₃₇ are independently selected from H, bromo, chloro, hydroxy, an amino, and an alkyl,
b) R₃₀ and R₃₄ are independently L-BRM; and
R₃₁ to R₃₃ and R₃₅-R₃₇ are independently selected from H, bromo, chloro, hydroxy, an amino, and alkyl; or
c) R₃₁ and R₃₅ are each L-BRM; and
R₃₂ to R₃₄, R₃₆ and R₃₇ are independently selected from H, bromo, chloro, hydroxy, an amino, and alkyl.

42-43. (canceled)

44. The compound of claim 38, wherein:
a) L1 and L2 are independently a linear linker having a backbone of 4 or more consecutive atoms, comprising one or more divalent linking moieties independently selected from —C₁₋₂₀-alkylene-, —NHCO—, —CONH—, —NH—, —⁺NH₂—, —NMe-, —⁺NMe₂-, —NHCONH—, —NHCSNH—, —(CH₂)ₚ—, —(OCH₂CH₂)ₚ—, —NHSO₂—, —SO₂NH—, —CO—, —SO₂—, —O—, and —S—, wherein each p is independently 1 to 10;
b) L1 and L2 are independently a linear linker having a backbone of 4 to 24 consecutive atoms, comprising 1 to 8 divalent linking moieties independently selected from —C₁₋₆-alkylene-, —NHCO—, —CONH—, —NH—, —⁺NH₂—, —NMe-, —⁺NMe₂-, —O(CH₂)ₚ—, —(OCH₂CH₂)ₚ—, —CO—, and —O—, wherein each p is independently 1 to 10; or
c) L1 and L2 are independently a linear linker having a backbone of 8 to 20 consecutive atoms, comprising 1 to 8 divalent linking moieties independently selected from —NHCO—, —CONH—, —NH—, —NMe-, —NH—, and —⁺NMe₂.

45-46. (canceled)

47. The compound of claim 38, wherein the compound is selected from:

| Cpd | |
|---|---|
| # | Structure |
| 8 | 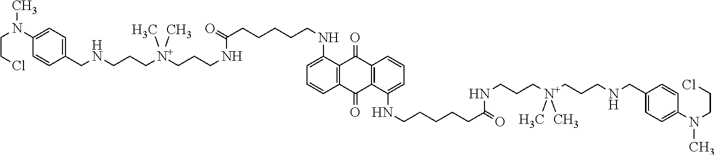 [SMILES: CN(CCCl)c1ccc(CNCCC[N+](C)(C)CCCNC(=O)CCCCCNc2cccc3c2C(=O)c2cccc(NCCCCCC(=O)NCCC[N+](C)(C)CCCNCc4ccc(N(C)CCCl)cc4)c2C3=O)cc1] |
| 14 | 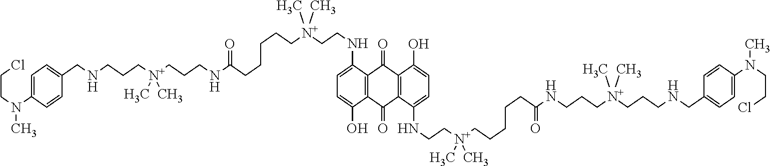 [SMILES: CN(CCCl)c1ccc(CNCCC[N+](C)(C)CCCNC(=O)CCCCC[N+](C)(C)CCNc2ccc(O)c3c2C(=O)c2c(O)ccc(NCC[N+](C)(C)CCCCCC(=O)NCCC[N+](C)(C)CCCNCc4ccc(N(C)CCCl)cc4)c2C3=O)cc1] |
| 20 | 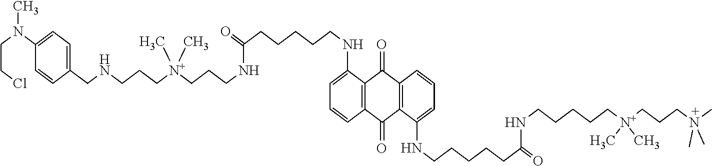 |
| 28 | 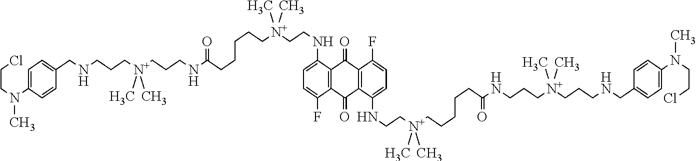 [SMILES: CN(CCCl)c1ccc(CNCCC[N+](C)(C)CCCNC(=O)CCCCC[N+](C)(C)CCNc2ccc(F)c3c2C(=O)c2c(F)ccc(NCC[N+](C)(C)CCCCCC(=O)NCCC[N+](C)(C)CCCNCc4ccc(N(C)CCCl)cc4)c2C3=O)cc1] |
| 29 | 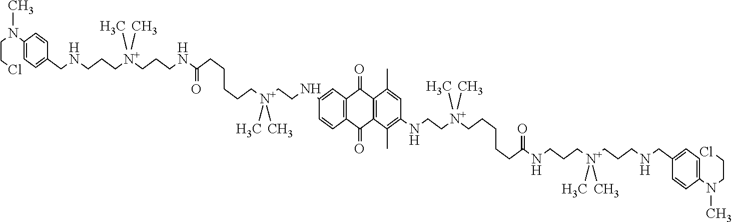 [SMILES: Cc1cc(NCC[N+](C)(C)CCCCCC(=O)NCCC[N+](C)(C)CCCNCc2ccc(N(C)CCCl)cc2)c(C)c2c1C(=O)c1cc(NCC[N+](C)(C)CCCCCC(=O)NCCC[N+](C)(C)CCCNCc3ccc(N(C)CCCl)cc3)ccc1C2=O] |
| 30 | 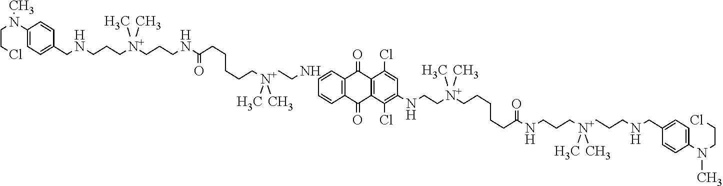 [SMILES: CN(CCCl)c1ccc(CNCCC[N+](C)(C)CCCNC(=O)CCCCC[N+](C)(C)CCNc2ccc3c(c2)C(=O)c2c(Cl)cc(NCC[N+](C)(C)CCCCCC(=O)NCCC[N+](C)(C)CCCNCc4ccc(N(C)CCCl)cc4)c(Cl)c2C3=O)cc1] |
| 31 | 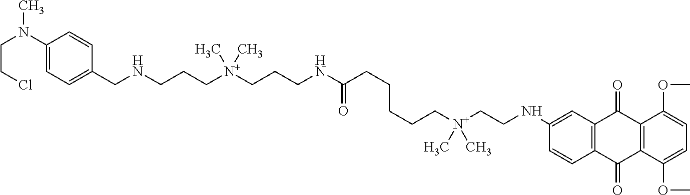 [SMILES: COc1ccc(OC)c2c1C(=O)c1ccc(NCC[N+](C)(C)CCCCCC(=O)NCCC[N+](C)(C)CCCNCc3ccc(N(C)CCCl)cc3)cc1C2=O] |
| 32 | 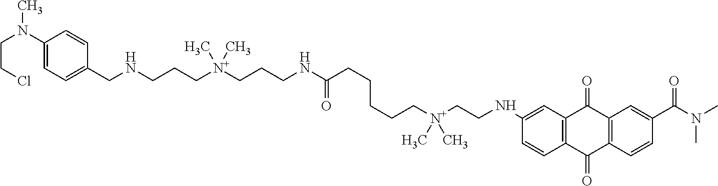 [SMILES: CN(C)C(=O)c1ccc2c(c1)C(=O)c1cc(NCC[N+](C)(C)CCCCCC(=O)NCCC[N+](C)(C)CCCNCc3ccc(N(C)CCCl)cc3)ccc1C2=O] |
| 33 | 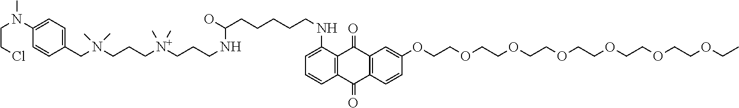 |

48-50. (canceled)

51. The compound of claim 5, wherein the compound is selected from:

| Cpd # | Structure |
|---|---|
| 24 | 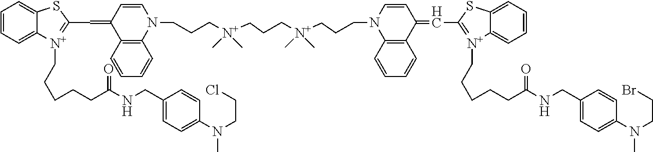 [SMILES: CN(CCCl)c1ccc(CNC(=O)CCCCC[n+]2c(C=C3C=CN(CCC[N+](C)(C)CCC[N+](C)(C)CCCN4C=CC(=Cc5sc6ccccc6[n+]5CCCCCC(=O)NCc5ccc(N(C)CCBr)cc5)c5ccccc54)c4ccccc43)sc3ccccc32)cc1] |
| 25 | 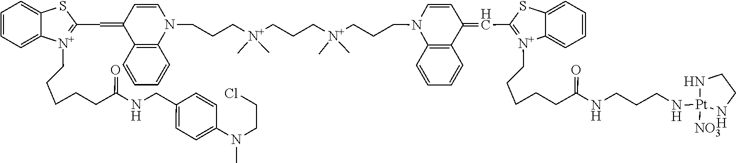 |

52. A method of detecting nucleic acids in a sample, the method comprising:
a) contacting a sample that contains a nucleic acid with a compound of claim 1;
b) incubating the contacted sample under conditions sufficient for the compound to associate with a nucleic acid in the sample to produce a nucleic acid complex that prevents the associated nucleic acid from being amplified;
c) extracting nucleic acid from the sample;
d) purifying the extracted nucleic acid; and
e) detecting a signal of an amplified nucleic acid from the extracted nucleic acid.

53-55. (canceled)

56. A kit for detecting nucleic acids in a sample, the kit comprising:
a) a compound of claim 1; and
b) one or more components selected from a buffer, a nucleic acid standard, a DNA or RNA ladder, a detergent, a matrix, and an instruction sheet concerning use of the kit for detecting nucleic acids in a sample.

57. (canceled)
